SEQUENCE LISTING (1 sequence)

SEQ ID NO: 1 - protein, 10 aa, Hepatitis C virus
  PEPTIDE (1)..(10): Xaa is Abu (aminobutyric acid)
EDVVXCSMSY

(19) United States
(12) Patent Application Publication
(10) Pub. No.: US 2013/0034522 A1
(43) Pub. Date: Feb. 7, 2013

(54) THERAPIES FOR TREATING HEPATITIS C VIRUS INFECTION

(71) Applicants: Maria Rosario, Mystic, CT (US); Nathalie Chauret, Quebec (CA); Shelley George, Wellesley, MA (US); Tara Lynn Kieffer, Brookline, MA (US); Margaret James Koziel, Needham, MA (US); Olivier Nicolas, Quebec (CA); Louise Proulx, Quebec (CA)

(72) Inventors: Maria Rosario, Mystic, CT (US); Nathalie Chauret, Quebec (CA); Shelley George, Wellesley, MA (US); Tara Lynn Kieffer, Brookline, MA (US); Margaret James Koziel, Needham, MA (US); Olivier Nicolas, Quebec (CA); Louise Proulx, Quebec (CA)

(73) Assignee: Vertex Pharmaceuticals Incorporated, Cambridge, MA (US)

(21) Appl. No.: 13/559,995

(22) Filed: Jul. 27, 2012

Related U.S. Application Data
(63) Continuation of application No. PCT/US11/22854, filed on Jan. 28, 2011.
(60) Provisional application No. 61/324,395, filed on Apr. 15, 2010.
(60) Provisional application No. 61/309,117, filed on Mar. 1, 2010.
(60) Provisional application No. 61/308,506, filed on Feb. 26, 2010.
(60) Provisional application No. 61/299,643, filed on Jan. 29, 2010.

(51) Int. Cl. A61K 31/497; A61K 31/7056; A61P 31/14; A61K 38/21

(52) U.S. Cl. 424/85.7; 514/255.05; 424/85.4; 514/43

(57) ABSTRACT

A method of improving the pharmacokinetics of VX-222 in a patient infected with HCV comprises co-administering VX-222 and VX-950 to the patient. A method of treating a patient infected with HCV comprises administering VX-222 and VX-950 to the patient, wherein VX-222 is in an amount of about 20 mg to about 400 mg, and wherein VX-950 is in an amount of about 100 mg to about 1,500 mg. A method of treating a patient infected with HCV comprises administering a therapeutically effective amount of VX-222, wherein VX-222 is administered at an amount of about 20 mg to about 2,000 mg once a day.

### RELATED APPLICATIONS

This application claims priority to U.S. Provisional Application Nos. 61/299,643 filed on Jan. 29, 2010, 61/308,506 filed on Feb. 26, 2010, 61/309,117 filed on Mar. 1, 2010, and 61/324,395 filed on Apr. 15, 2010. The entire teachings of these applications are incorporated herein by reference.

Incorporated herein by reference is a Sequence Listing named “10-112USWCN1_ST25.txt” created Oct. 3, 2012, that is 913 bytes. The sequence listing information recorded in computer readable form is identical to the written (on paper or compact disc) sequence listing and does not include any new matter which goes beyond the disclosure of the application as filed.

### TECHNICAL FIELD OF THE INVENTION

The present invention relates to methods for treating Hepatitis C virus infections.

### BACKGROUND OF THE INVENTION

Infection by Hepatitis C virus (“HCV”) is a compelling human medical problem. HCV is recognized as the causative agent for most cases of non-A, non-B hepatitis, with an estimated human sero-prevalence of 3% globally (see, e.g., A. Alberti et al., “Natural History of Hepatitis C,” J. Hepatology, 31 (Suppl. 1), 17-24 (1999)). Nearly four million individuals may be infected in the United States alone (see, e.g., M. J. Alter et al., “The Epidemiology of Viral Hepatitis in the United States, Gastroenterol. Clin. North Am., 23, 437-455 (1994); M. J. Alter, “Hepatitis C Virus Infection in the United States,” J. Hepatology, 31 (Suppl. 1), 88-91 (1999)).

Of persons who become infected with HCV, 20-25% may be able to clear the virus after the acute infection, but 75-80% will develop chronic Hepatitis C infection. (See, e.g., preface, Frontiers in Viral Hepatitis, Ed. R F Schinazi, J-P Sommadossi, and C M Rice, p. xi., Elsevier (2003)). This usually results in recurrent and progressively worsening liver inflammation, which often leads to more severe disease states such as cirrhosis and hepatocellular carcinoma (see, e.g., M. C. Kew, “Hepatitis C and Hepatocellular Carcinoma”, FEMS Microbiology Reviews, 14, 211-220 (1994); I. Saito et. al., “Hepatitis C Virus Infection is Associated with the Development of Hepatocellular Carcinoma,” Proc. Natl. Acad. Sci. USA, 87, 6547-6549 (1990)). Unfortunately, there are no broadly effective treatments for the debilitating progression of chronic HCV.

The HCV genome encodes a polyprotein of 3010-3033 amino acids (see, e.g., Q. L. Choo, et. al., “Genetic Organization and Diversity of the Hepatitis C Virus,” Proc. Natl. Acad. Sci. USA, 88, 2451-2455 (1991); N. Kato et al., “Molecular Cloning of the Human Hepatitis C Virus Genome From Japanese Patients with Non-A, Non-B Hepatitis,” Proc. Natl. Acad. Sci. USA, 87, 9524-9528 (1990); A. Takamizawa et al., “Structure and Organization of the Hepatitis C Virus Genome Isolated From Human Carriers,” J. Virol., 65, 1105-1113 (1991)0. The HCV nonstructural (NS) proteins are presumed to provide the essential catalytic machinery for viral replication. The NS proteins are derived by proteolytic cleavage of the polyprotein (see, e.g., R. Bartenschlager et. al., “Nonstructural Protein 3 of the Hepatitis C Virus Encodes a Serine-Type Proteinase Required for Cleavage at the NS3/4 and NS4/5 Junctions,” J. Virol., 67, 3835-3844 (1993); A. Grakoui et. al., “Characterization of the Hepatitis C Virus-Encoded Serine Proteinase: Determination of Proteinase-Dependent Polyprotein Cleavage Sites,” J. Virol., 67, 2832-2843 (1993); A. Grakoui et. al., “Expression and Identification of Hepatitis C Virus Polyprotein Cleavage Products,” J. Virol., 67, 1385-1395 (1993); L. Tomei et. al., “NS3 is a serine protease required for processing of hepatitis C virus polyprotein”, J. Virol., 67, 4017-4026 (1993)).

The HCV NS protein 3 (NS3) contains a serine protease activity that helps process the majority of the viral enzymes, and is thus considered essential for viral replication and infectivity. It is known that mutations in the yellow fever virus NS3 protease decreases viral infectivity (see, e.g., Chambers, T. J. et. al., “Evidence that the N-terminal Domain of Nonstructural Protein NS3 From Yellow Fever Virus is a Serine Protease Responsible for Site-Specific Cleavages in the Viral Polyprotein”, Proc. Natl. Acad. Sci. USA, 87, 8898-8902 (1990)). The first 181 amino acids of NS3 (residues 1027-1207 of the viral polyprotein) have been shown to contain the serine protease domain of NS3 that processes all four downstream sites of the HCV polyprotein (see, e.g., C. Lin et al., “Hepatitis C Virus NS3 Serine Proteinase: Trans-Cleavage Requirements and Processing Kinetics”, J. Virol., 68, 8147-8157 (1994)).

The HCV NS3 serine protease and its associated cofactor, NS4A, help process all of the viral enzymes, and is thus considered essential for viral replication. This processing appears to be analogous to that carried out by the human immunodeficiency virus aspartyl protease, which is also involved in viral enzyme processing. HIV protease inhibitors, which inhibit viral protein processing are potent antiviral agents in man, indicating that interrupting this stage of the viral life cycle results in therapeutically active agents. Consequently it is an attractive target for drug discovery.

There are not currently any satisfactory anti-HCV agents or treatments. Until recently, the only established therapy for HCV disease was interferon treatment. The first approved therapy for HCV infection was treatment with standard (non-pegylated) interferon-alfa. However, interferons have significant side effects (see, e.g., M. A. Wlaker et al., “Hepatitis C Virus: An Overview of Current Approaches and Progress,” DDT, 4, 518-29 (1999); D. Moradpour et al., “Current and Evolving Therapies for Hepatitis C,” Eur. J. Gastroenterol. Hepatol., 11, 1199-1202 (1999); H. L. A. Janssen et al. “Suicide Associated with Alfa-Interferon Therapy for Chronic Viral Hepatitis,” J. Hepatol., 21, 241-243 (1994); P. F. Renault et al., “Side Effects of Alpha Interferon,” Seminars in Liver Disease, 9, 273-277, (1989)) and interferon alfa monotherapy induces long term remission in only a fraction (˜25%) of cases (see, e.g., O. Weiland, “Interferon Therapy in Chronic Hepatitis C Virus Infection”, FEMS Microbiol. Rev., 14, 279-288 (1994)). The addition of ribavirin to the treatment regimen increases response rates slightly. Recent introductions of the pegylated forms of interferon (PEG-INTRON® and PEGASYS®), which has also been combined with ribavirin have resulted in only modest improvements in remission rates and only partial reductions in side effects. The current standard of care is a treatment regimen lasting 24-48 weeks, depending on prognostic factors such as HCV genotype and demonstration of initial response to therapy. Moreover, the prospects for effective anti-HCV vaccines remain uncertain.

Thus, there is a need for anti-HCV therapies and appropriate dose regimens for anti-HCV compounds.

HCV and other diseases and disorders are associated with liver damage. There is also a need for therapies and appropriate dose regimens for treating liver damage.

### SUMMARY OF THE INVENTION

The present invention generally provides a treatment for Hepatitis C virus (HCV) infections. The invention also generally provides for the prevention of the clinical sequelae of Hepatitis C viral infections.

In one aspect, the present invention is directed to a method of improving the pharmacokinetics of VX-222 in a patient infected with HCV. The method comprises co-administering VX-222 and VX-950 to the patient.

In another aspect, the present invention is directed to a method of increasing the exposure of VX-222 in the plasma of a patient infected with HCV. The method comprises administering VX-222 and VX-950 to the patient.

In another aspect, the present invention is directed to a method of treating a patient infected with HCV. The method comprises administering VX-222 and VX-950 to the patient, wherein VX-222 is in an amount of about 20 mg to about 400 mg, and wherein VX-950 is in an amount of about 100 mg to about 1500 mg.

In yet another aspect, the present invention is directed to a method of treating a patient infected with HCV, comprising administering a therapeutically effective amount of VX-222, wherein VX-222 is administered at an amount of about 20 mg to about 2,000 mg once a day.

In yet another aspect, the present invention is directed a pharmaceutically acceptable composition, comprising: a) VX-222 in an amount of about 20 mg to about 400 mg; and b) VX-950 is in an amount of about 100 mg to about 1500 mg.

The present invention also provides use of VX-222 and VX-950 in the manufacture of a medicament for increasing bioavailability of VX-222 in a patient infected with HCV.

The present invention also provides use of VX-222 and VX-950 in the manufacture of a medicament for increasing bioavailability or exposure of VX-222 in the plasma of a patient infected with HCV.

The present invention also provides use of VX-222 and VX-950 in the manufacture of a medicament for treating a patient infected with HCV, wherein VX-222 is in an amount of about 20 mg to about 400 mg, and wherein VX-950 is in an amount of about 100 mg to about 1500 mg.

The present invention also provides use of VX-222 in the manufacture of a medicament for treating a patient infected with HCV, wherein VX-222 is administered at an amount of about 20 mg to about 2,000 mg, or about 50 mg to about 2,000 mg, once a day.

### BRIEF DESCRIPTION OF THE FIGURES

FIGS. 1 and 2 are charts showing the study design of certain embodiments of the invention.

FIGS. 3-8 are charts showing the study results of one embodiment of the invention.

FIG. 9 shows a graph showing plasma levels of a prodrug of Compound 1 and its conversion into an active metabolite after dosing of the prodrug.

### DETAILED DESCRIPTION OF THE INVENTION

This invention relates to specific doses and dosage regimens for administering VX-222. For the purpose of this invention, VX-222 includes Compound 1 and pharmaceutically acceptable salts, solvates, and prodrugs thereof, and also pharmaceutically acceptable solvates of a prodrug of Compound 1, wherein Compound 1 is represented by the following structural formula:

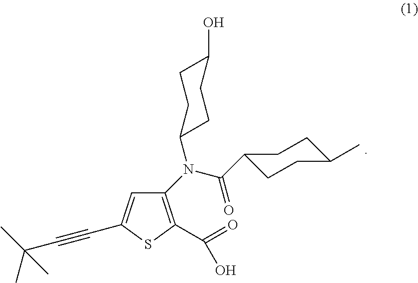 [SMILES: CC1CCC(C(=O)N(c2cc(C#CC(C)(C)C)sc2C(=O)O)C2CCC(O)CC2)CC1]

VX-222 is a NS5B polymerase inhibitor, and described in WO 2008/058393.

This invention also relates to specific doses and dosage regimens for administering VX-950. VX-950 is a competitive, reversible peptidomimetic NS3/4A protease inhibitor with a steady state binding constant (ki*) of 7 nM. See, e.g., WO 02/018369. For the purpose of this invention, VX-950 includes Compound 2 and pharmaceutically acceptable salts and prodrugs of Compound 2, wherein Compound 2 is represented by the following structural formula:

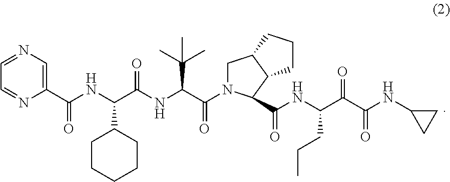 [SMILES: CCC[C@H](NC(=O)[C@@H]1[C@H]2CCC[C@H]2CN1C(=O)[C@@H](NC(=O)[C@@H](NC(=O)c1cnccn1)C1CCCCC1)C(C)(C)C)C(=O)C(=O)NC1CC1]

VX-950 is described in PCT Publication Numbers WO 02/018369, WO 2006/050250, and WO 2008144072. Other descriptions of VX-950 can be found in PCT Publication Numbers WO 07/098,270 and WO 08/106,151.

As used herein, the phrase “pharmaceutically acceptable salt(s)” refers to the salts that are safe and effective for treatment of HCV infections. Pharmaceutically acceptable acid addition salts include, but are not limited to, hydrochloride, hydrobromide, hydroiodide, nitrate, sulfate, bisulfate, phosphate, acid phosphate, isonicotinate, acetate, and lactate salts. Pharmaceutically acceptable salts with various amino acids can also be used, and use of these amino acid salts is also within the scope of this invention. Suitable base salts include, but are not limited to, aluminum, calcium, lithium, magnesium, potassium, sodium, zinc, and diethanolamine salts. For a review on pharmaceutically acceptable salts, see Berge et al., J. Pharm. Sci., 66, 1-19 (1977), the contents of which are incorporated herein by reference.

Specific examples of pharmaceutically acceptable salts of Compound 1 are described in WO 2008/058393, such as salts derived from amino acids (e.g. L-arginine, L-Lysine), salts derived from appropriate bases include alkali metals (e.g. sodium, lithium, potassium), alkaline earth metals (e.g. calcium, magnesium), ammonium, NR₄⁺ (where R is C₁₋₄ alkyl) salts, choline and tromethamine salts. In one embodiment, the pharmaceutically acceptable salt is a sodium salt. In another embodiment, the pharmaceutically acceptable salt is a lithium salt. In yet another embodiment, the pharmaceutically acceptable salt is a potassium salt. In yet another embodiment, the pharmaceutically acceptable salt is a tromethamine salt. In yet another embodiment, the pharmaceutically acceptable salt is an L-arginine salt.

As used herein, the phrase a “pharmaceutically acceptable prodrug” of Compound 1 refers to a compound that may be converted under physiological conditions or by solvolysis to Compound 1 or to a pharmaceutically acceptable salt of Compound 1 prior to exhibiting its pharmacological effect in the treatment of HCV infections. As used herein, the phrase a “pharmaceutically acceptable prodrug” of Compound 2 refers to a compound that may be converted under physiological conditions or by solvolysis to Compound 2 or to a pharmaceutically acceptable salt of Compound 2 prior to exhibiting its pharmacological effect in the treatment of HCV infections. Typically, the prodrugs are formulated with the objectives of improved chemical stability, improved patient acceptance and compliance, improved bioavailability, prolonged duration of action, improved organ selectivity, improved formulation (e.g., increased hydrosolubility), or decreased side effects (e.g., toxicity).

A pharmaceutically acceptable prodrug can be readily prepared using methods known in the art, such as those described in Burger's Medicinal Chemistry and Drug Chemistry, Vol. 1, 172-178 and 949-982, John Wiley & Sons (1995). See also Bertolini et al., J. Med. Chem., 40, 2011-2016 (1997); Shan et al., J. Pharm. Sci., 86(7), 765-767 (1997); Bagshawe, Drug Dev. Res., 34, 220-230 (1995); Bodor, Advances in Drug Res., 13, 224-331 (1984); Bundgaard, Design of Prodrugs, Elsevier Press (1985); and Larsen, Design and Application of Prodrugs, Drug Design and Development (Krogsgaard-Larsen et al., eds.), Harwood Academic Publishers (1991).

Specific examples of prodrugs of Compound 1 include those described in U.S. Ser. No. 61/359,164 filed on Jun. 28, 2010:

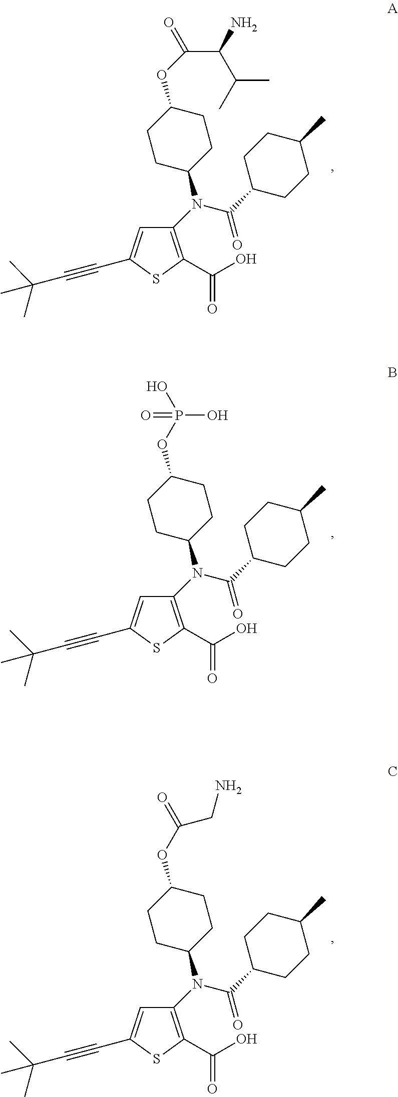

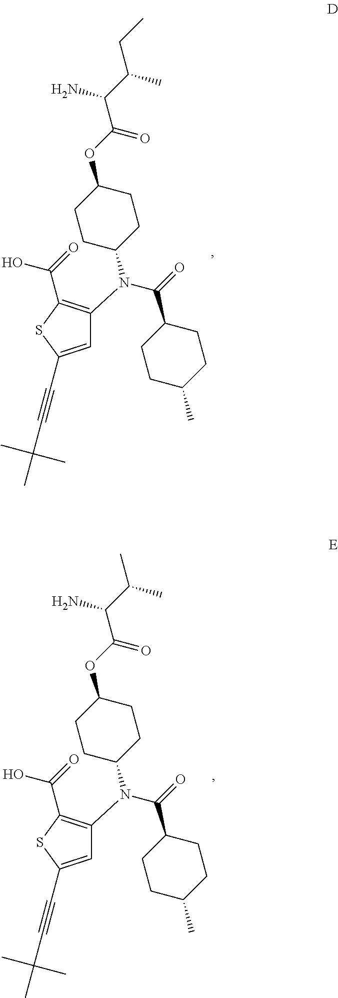

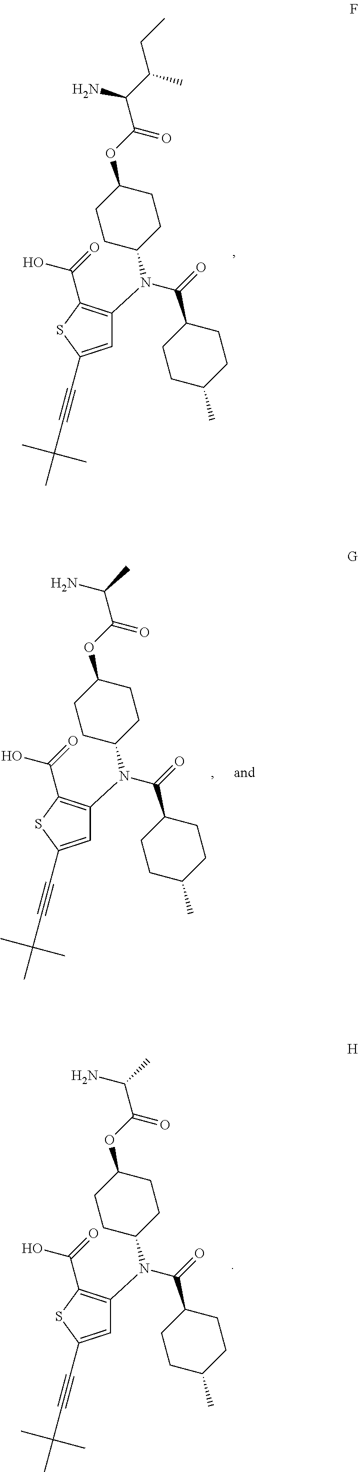 [SMILES: CC[C@H](C)[C@H](N)C(=O)O[C@H]1CC[C@H](N(c2cc(C#CC(C)(C)C)sc2C(=O)O)C(=O)[C@H]2CC[C@H](C)CC2)CC1 ; C[C@H](N)C(=O)O[C@H]1CC[C@H](N(c2cc(C#CC(C)(C)C)sc2C(=O)O)C(=O)[C@H]2CC[C@H](C)CC2)CC1 ; C[C@@H](N)C(=O)O[C@H]1CC[C@H](N(c2cc(C#CC(C)(C)C)sc2C(=O)O)C(=O)[C@H]2CC[C@H](C)CC2)CC1]

It will further be appreciated by those skilled in the art that the compounds described herein can exist in different solvate forms, for example hydrates, and yet retains the biological effectiveness. Such solvates may also form when solvent molecules are incorporated into the crystalline lattice structure of the compound molecule during the crystallization process. As used herein, the phrase a “pharmaceutically acceptable solvate” of Compound 1 refers to a pharmaceutically acceptable solvate form of Compound 1 that contains solvent molecule(s) and retains the biological effectiveness of Compound 1. As used herein, the phrase a “pharmaceutically acceptable solvate” of a prodrug of Compound 1, refers to a pharmaceutically acceptable solvate form of a prodrug of Compound 1 that contains solvent molecule(s) and retains the biological effectiveness of Compound 1.

Compounds that differ from Compound 1 and Compound 2 only in the presence of one or more isotopically enriched atoms are covered in the invention. For example, compounds having the present structures except for the replacement of hydrogen by deuterium or tritium, or the replacement of a carbon by a ¹³C- or ¹⁴C-enriched carbon are within the scope of this invention. Certain examples of isotopically enriched Compound 2 can be found in WO 2007/109080 and Maltais et al., J. of Medicinal Chemistry, “In Vitro and In Vivo Isotope Effects with Hepatitis C Protease Inhibitors: Enhanced Plasma Exposure of Deuterated Telaprevir versus Telaprevir in Rats” 2009; 52(24):7993-8001.

Compound 1 and Compound 2 may each independently contain one or more asymmetric carbon atoms and thus may occur as racemates and racemic mixtures, single enantiomers, diastereomeric mixtures and individual diastereomers. All such isomeric forms of these compounds are expressly included in the present invention. Each stereogenic carbon may be of the R or S configuration. The D- and L-isomers at the N-propyl side chain of Compound 2 are expressly included within the scope of this invention.

It will be appreciated by those skilled in the art that the compounds described herein can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

In one aspect, for any one of the embodiments of the invention described below, VX-222 is Compound 1, or a pharmaceutically acceptable salt, solvate or prodrug thereof, or a solvate of a prodrug of Compound 1; and VX-950 is Compound 2, or a pharmaceutically acceptable salt or prodrug thereof.

In another aspect, for any one of the embodiments of the invention described below, VX-222 is Compound 1, or a pharmaceutically acceptable salt, solvate or prodrug thereof, or a solvate of a prodrug of Compound 1; and VX-950 is Compound 2 or a pharmaceutically acceptable salt thereof.

In yet another aspect, for any one of the embodiments of the invention described below, VX-222 is Compound 1, or a pharmaceutically acceptable salt, solvate or prodrug thereof, or a solvate of a prodrug of Compound 1; and VX-950 is Compound 2.

In yet another aspect, for any one of the embodiments of the invention described below, VX-222 is Compound 1, or a pharmaceutically acceptable salt or solvate, or prodrug thereof; and VX-950 is Compound 2, or a pharmaceutically acceptable salt or prodrug thereof.

In yet another aspect, for any one of the embodiments of the invention described below, VX-222 is Compound 1, or a pharmaceutically acceptable salt or solvate, or prodrug thereof; and VX-950 is Compound 2 or a pharmaceutically acceptable salt thereof.

In yet another aspect, for any one of the embodiments of the invention described below, VX-222 is Compound 1, or a pharmaceutically acceptable salt or solvate, or prodrug thereof; and VX-950 is Compound 2.

In yet another aspect, for any one of the embodiments of the invention described below, VX-222 is Compound 1, or a pharmaceutically acceptable salt or solvate thereof and VX-950 is Compound 2, or a pharmaceutically acceptable salt or prodrug thereof.

In yet another aspect, for any one of the embodiments of the invention described below, VX-222 is Compound 1, or a pharmaceutically acceptable salt or solvate thereof and VX-950 is Compound 2 or a pharmaceutically acceptable salt thereof.

In yet another aspect, for any one of the embodiments of the invention described below, VX-222 is Compound 1, or a pharmaceutically acceptable salt or solvate thereof and VX-950 is Compound 2.

In yet another aspect, for any one of the embodiments of the invention described below, VX-222 is Compound 1 or a pharmaceutically acceptable salt thereof and VX-950 is Compound 2, or a pharmaceutically acceptable salt or prodrug thereof.

In yet another aspect, for any one of the embodiments of the invention described below, VX-222 is Compound 1 or a pharmaceutically acceptable salt thereof; and VX-950 is Compound 2 or a pharmaceutically acceptable salt thereof.

In yet another aspect, for any one of the embodiments of the invention described below, VX-222 is Compound 1 or a pharmaceutically acceptable salt thereof; and VX-950 is Compound 2.

In yet another aspect, for any one of the embodiments of the invention described below, VX-222 is Compound 1; and VX-950 is Compound 2, or a pharmaceutically acceptable salt or prodrug thereof.

In yet another aspect, for any one of the embodiments of the invention described below, VX-222 is Compound 1; and VX-950 is Compound 2 or a pharmaceutically acceptable salt thereof.

In yet another aspect, for any one of the embodiments of the invention described below, VX-222 is Compound 1; and VX-950 is Compound 2.

In one embodiment, the present invention is directed to methods of improving the pharmacokinetics of VX-222 in a patient infected with HCV. The method comprises co-administering VX-222 and VX-950 to the patient. The improved pharmacokinetics of VX-222 include an increase in the exposure of VX-222 in the plasma, blood, or liver of the patient. In another embodiment, the present invention is directed to methods of increasing the exposure of VX-222 in the plasma of a patient infected with HCV. The exposure of VX-222, for example, in the plasma, can be measured by a trough level (C_{trough}), an average plasma concentration (C_{avg}), the maximum plasma concentration (Cₘₐₓ), or an AUC (area under curve) value, of VX-222. As used herein, the term “trough level” (C_{trough}) refers to the concentration of a drug in plasma just before the next dose, or the minimum drug concentration between two doses. As used herein, the term “AUC” refers to the area under the plasma (serum, or blood) concentration versus time curve. In a specific embodiment, the exposure of VX-222 is indicated with a value of AUC₀₋₁₂ (from zero to 12 hours). In another specific embodiment, the exposure of VX-222 is indicated with a value of AUC₀₋₂₄ (from zero to 24 hours).

The increase of the exposure of VX-222 by co-administration of VX-950 can be determined by comparison of the exposure of VX-222 administered without VX-950 with that of VX-222 co-administered with VX-950. In one specific embodiment, the exposure of VX-222 is increased by about a two- to six-fold compared to that of VX-222 administered without VX-950. In one specific embodiment, the exposure of VX-222 is increased by about a two- to five-fold compared to that of VX-222 administered without VX-950. In one specific embodiment, the exposure of VX-222 is increased by about a two- or three-fold compared to that of VX-222 administered without VX-950. In some specific embodiments, the increase is in the plasma exposure of VX-222.

In the invention, the amount of VX-950 at each administration can be from about 100 mg to about 1,500 mg, from about 300 mg to about 1,500 mg, from about 500 mg to about 1,500 mg, from about 300 mg to about 1,250 mg, about 450 mg, about 750 mg, or about 1,250 mg. In some specific embodiments, VX-950 is in an amount of about 750 mg at each administration. In some specific embodiments, VX-950 is in an amount of about 1,125 mg at each administration. Suitable examples of the amounts of VX-950 are described in WO 2008/144072 and WO 06/050250, the entire teachings of which are incorporated herein by reference.

In the invention, the amount of VX-222 at each administration can be from about from about 20 mg to about 2,000 mg, from about 50 mg to about 2,000 mg, from about 100 mg to about 1,500 mg, from about 100 mg to about 1,250 mg, from about 100 mg to about 1,000 mg, about 100 mg, about 250 mg, about 400 mg, or about 750 mg. In some specific embodiments, VX-222 is in an amount of about 100 mg at each administration. In some specific embodiments, VX-222 is in an amount of about 250 mg at each administration. In some specific embodiments, VX-222 is in an amount of about 400 mg at each administration. In some specific embodiments, VX-222 is in an amount of about 750 mg at each administration. In some specific embodiments, VX-222 is in an amount of about 1,000 mg at each administration. In some specific embodiments, VX-222 is in an amount of about 1,500 mg at each administration. In some specific embodiments, VX-222 is in an amount of about 500 mg at each administration. In some specific embodiments, VX-222 is in an amount of about 1,125 mg at each administration. In some specific embodiments, VX-222 is in an amount of about 1,250 mg at each administration.

In yet another embodiment, the present invention is directed to methods of treating a patient infected with HCV, comprising administering of VX-222 and VX-950 to the patient. In a specific embodiment, VX-222 is in an amount of from about 20 mg to about 2,000 mg, such as from about 50 mg to about 1,500 mg, at each administration, and VX-950 is in an amount of from about 100 mg to about 1,500 mg, such as from about 300 mg to about 1,500 mg, at each administration. In another specific embodiment, VX-222 is in an amount of from about 20 mg to about 400 mg, such as from about 50 mg to about 400 mg, at each administration, and VX-950 is in an amount of from about 100 mg to about 1500 mg at each administration. In yet another specific embodiment, VX-222 is in an amount of equal to, or greater than, 20 mg and less than 400 mg at each administration. In yet another specific embodiment, VX-222 is in an amount of from about 20 mg to about 300 mg at each administration. In yet another specific embodiment, VX-222 is in an amount of from about 50 mg to about 300 mg at each administration. In yet another specific embodiment, VX-222 is in an amount of about 100 mg at each administration. In yet another specific embodiment, VX-222 is in an amount of about 400 mg at each administration. In yet another specific embodiment, VX-950 is in an amount of from about 300 mg to about 1,500 mg at each administration. In yet another specific embodiment, VX-950 is in an amount of from about 500 mg to about 1,500 mg at each administration. In yet another specific embodiment, VX-950 is in an amount of about 750 mg at each administration. In yet another specific embodiment, VX-950 is in an amount of about 1,125 mg at each administration.

In yet another embodiment, the present invention is directed to methods of treating a patient infected with HCV, comprising administering VX-222, wherein VX-222 is administered at an amount of about 20 mg to about 2,000 mg. Specifically, the amount of VX-222 can be from about 100 mg to about 1,500 mg, from about 100 mg to about 1,250 mg, from about 100 mg to about 1,000 mg, about 100 mg, about 250 mg, about 400 mg, about 500 mg, about 750 mg, about 1000 mg, about 1125 mg, or about 1250 mg, at each administration. In some embodiments, VX-222 is administered once a day. In a specific embodiment, the methods comprise administering VX-222 at an amount of about 50 mg to about 2,000 mg once a day. In some specific embodiments, the amount of VX-222 administered is about 100 mg once a day. In some specific embodiments, the amount of VX-222 administered is about 250 mg once a day. In some specific embodiments, the amount of VX-222 administered is about 400 mg once a day. In some specific embodiments, the amount of VX-222 administered is about 500 mg once a day. In some specific embodiments, the amount of VX-222 administered is about 750 mg once a day. In some specific embodiments, the amount of VX-222 administered is about 1,000 mg once a day. In some specific embodiments, the amount of VX-222 administered is about 1,250 mg once a day. In some specific embodiments, the amount of VX-222 administered is about 1,125 mg once a day. In some specific embodiments, the amount of VX-222 administered is about 1,500 mg once a day.

In a specific embodiment, the methods of treating a patient infected with HCV by employing administration of VX-222 from about 20 mg to about 2,000 mg (or from about 50 mg to about 2,000 mg, or any specific dosage regimens discussed above) once a day and further administration of one or more additional HCV drugs other than VX-222. Suitable examples of the additional HCV drugs are described below in detail, which include VX-950, interferon, and ribavirin. In a further specific embodiment, VX-950 is co-administered. Typical examples of the amounts of VX-950 are as described above. In another further specific embodiment, an interferon (e.g., pegylated interferon, such as pegylated interferon alpha-2a or pegylated interferon alpha-2b) with or without ribavirin is co-administered. In another further specific embodiment, VX-950; an interferon (e.g., pegylated interferon, such as pegylated interferon alpha-2a or pegylated interferon alpha-2b); and ribavirin is co-administered.

In yet another embodiment, the present invention provides pharmaceutically acceptable compositions comprising VX-222 in an amount of about 20 mg to about 2,000 mg; and VX-950 in an amount of from about 100 mg to about 1,500 mg. Optionally, a pharmaceutically acceptable carrier can also be included. In a specific embodiment, the present invention provides pharmaceutically acceptable compositions comprising VX-222 in an amount of from about 20 mg to about 1,500 mg, or from about 50 mg to about 1,500 mg. In another specific embodiment, the amount of VX-950 in these pharmaceutical compositions is from about 300 mg to about 1500 mg, from about 300 mg to about 1250 mg, from about 300 mg to about 1,000 mg, from about 300 mg to about 750 mg, or about 375 mg. In another specific embodiment, the amount of VX-222 in these pharmaceutical compositions is equal to, or greater than 50 mg and less than 400 mg, from about 50 mg to about 300 mg, about 50 mg, about 100 mg, or about 200 mg. Each of these pharmaceutical compositions can be administered, e.g., once, twice, or three times per day. Each of these compositions can be in one or more dosage forms (e.g., ampule, capsule, cream, emulsion, fluid, grain, drop, injection, suspension, tablet, powder). Each of these pharmaceutical compositions can be administered by one or more routes (e.g., orally, by infusion, by injection, topically, or parenterally) as considered appropriate by a skilled person in the art and depending on the dosage form.

In general, in the methods of the invention described above, each of VX-222 and VX-950 can independently be administered once a day (QD), twice a day (e.g., BID; q12h), three times a day (e.g., TID; q8h), or four times a day. Each of VX-222 and VX-950 may independently be administered with or without food.

In some embodiments, the methods of the invention include administering to the patient VX-950 (a) in an amount of about 450 mg each time, 3 times per day, once every 8 hours; (b) in an amount of about 750 mg each administration, 3 times per day, once every 8 hours; (c) in an amount of about 1,125 mg each administration, 2 times per day, once every 12 hours; or (d) in an amount of about 1250 mg each time, 2 times per day, once every 1 hours.

In some embodiments, the methods of the invention includes administering to the patient an oral dose of a composition comprising VX-950, wherein said dose provides to the patient an average plasma concentration (C_{avg}) of VX-950 of at least about 750 ng/mL after the administration. In some embodiments, the C_{avg} of VX-950 is about 1000 ng/mL or about 1250 ng/mL. In some embodiments, said dose essentially contains about 750 mg of VX-950. In some embodiments, the C_{avg} is obtained or attained within 3 hours (e.g., 2 hours or 1 hour) after administration of VX-950. In some embodiments, the C_{avg} of VX-950 is maintained over about 24 hours (e.g., 5 weeks or 12 weeks).

In some embodiments, the methods of the invention includes administering to the patient VX-950, wherein the trough VX-950 plasma level is maintained at a minimum of about 750, 800, 900, or 1000 ng/mL over a 24 hour period. Without being bound by theory, trough levels of more than about 1500 ng/mL are thought to be not required by this invention. Accordingly, trough levels of about 750, 800, 900, 1000 ng/mL to about 1500 ng/mL (particularly 1000 to about 1500) are within the scope of this invention.

Ideally, when a method of this invention involves treating a patient infected with HCV, the method involves achieving, relatively rapidly, a therapeutically effective plasma concentration of VX-950 and then maintaining the trough level such that an effective therapeutic response is achieved. An effective therapeutic response is, preferably, one or both of a) achieving a sustained viral response; and b) achieving undetectable HCV RNA in the plasma by at least 12 weeks (12 weeks or more). As used herein, HCV RNA being “undetectable” means that the HCV RNA is present in less than 10 IU/mL as determined by assays currently commercially available, and preferably as determined by the Roche COBAS TaqMan™ HCV/HPS assay.

The relatively rapid drop in plasma concentration may be obtained by administering a loading dose to a patient. In one embodiment, the loading dose is about 1250 mg of VX-950.

In some embodiments, the methods of the invention comprise co-administering VX-950, and VX-950 is in a dosage form containing about 750 mg of VX-950 (e.g., two tablets of about 375 mg of VX-950) and the dosage form is administered three times per day, e.g., once every 8 hours (i.e., q8h). In some embodiments, the methods of the invention comprises administering VX-950, and VX-950 is in a dosage form containing about 1125 mg of VX-950 (e.g., three tablets of about 375 mg of VX-950) and the dosage form is administered two times per day, e.g., once every 12 hours (i.e., q12h).

In the invention, VX-222 and any additional HCV drugs (such as VX-950; an interferon (e.g., pegylated interferon, such as pegylated interferon alpha-2a or pegylated interferon alpha-2b) with or without ribavirin; or VX-950, an interferon (e.g., pegylated interferon, such as pegylated interferon alpha-2a or pegylated interferon alpha-2b), and ribavirin) can independently be administered over the entire treatment period. In these embodiments, the VX-222 treatment period and the treatment period of the additional HCV drug(s) are the same.

Alternatively, in some embodiments, VX-222 and any additional HCV drugs (such as VX-950; an interferon (e.g., pegylated interferon, such as pegylated interferon alpha-2a or pegylated interferon alpha-2b) with or without ribavirin; or VX-950, an interferon (e.g., pegylated interferon, such as pegylated interferon alpha-2a or pegylated interferon alpha-2b), and ribavirin) can independently be administered over two phases, an initial phase and a secondary phase. Each of VX-222 and any additional HCV drugs may be administered in either the initial or secondary phase, or in both phases. In some embodiments, VX-222 is administered only in the initial phase, and interferon is administered in both of the initial and secondary phases. Alternatively, in some other embodiments, VX-222 is administered only in the secondary phase, and interferon is administered in both of the initial and secondary phases. In some embodiments, VX-222 and VX-950 are co-administered, and VX-222 and VX-950 are administered only in the initial phase, or only in the secondary phase. In some embodiments, VX-222, VX-950, an interferon (e.g., pegylated interferon, such as pegylated interferon alpha-2a or pegylated interferon alpha-2b), and ribavirin are co-administered, and VX-222 and VX-950 are administered only in the initial phase, and the interferon and ribavirin are administered in both of the initial and secondary phases.

Suitable, specific examples of duration of the initial and secondary phases can be found in WO 2008/144072. For instance the initial phase can be a period of at least about 4 weeks, or between about 4 weeks and about 24 weeks (e.g., about 4 weeks, about 6 weeks, about 8 weeks, about 10 weeks, about 12 weeks, about 16 weeks, about 20 weeks, about 24 weeks, etc.), and the secondary phase can be at least about 12 weeks, e.g., the secondary phase can be about 12 weeks to about 36 weeks. In certain embodiments, the secondary phase is about 12 weeks. In other embodiments, the secondary phase is about 24 weeks. In still other embodiments, the secondary phase is about 36 weeks. In certain embodiments, the sum of the initial and secondary phase is about 24 weeks to about 48 weeks (such as about 24, 36, or 48 weeks). In some embodiments, the initial and secondary phases can be identical in duration.

In some embodiments, the methods of the invention comprise co-administering VX-222 and interferon independently for a period from about 4 weeks to about 12 weeks (e.g., about 4, 6, 8 or 12 weeks), for a period from about 20 weeks to about 28 weeks (e.g., about 20, 24, or 28 weeks), or for a period of from about 8 weeks to about 24 weeks (e.g., about 8, 12, 16 or 24 weeks). In one aspect of each of these embodiments, the administration of VX-222 and interferon independently (initial phase) is followed by administration of interferon (without VX-222) (secondary phase) for a period from about 4 weeks to about 36 weeks (e.g., from about 8 weeks to about 36 weeks, from about 8 weeks to about 24 weeks, from about 4 weeks to about 24 weeks). Specific exemplary regimens include: administering VX-222 and interferon independently for about 8 weeks followed by administering interferon (without VX-222) for about 16 weeks for a total treatment regimen of about 24 weeks; administering VX-222 and interferon independently for about 12 weeks followed by administering interferon (without VX-222) for about 12 weeks for a total treatment regimen of about 24 weeks. In such regimens, optionally is provided administration of ribavirin for all (for both initial and secondary phases), or a part of each regimen (e.g., only for the initial phase or only for the secondary phase).

In some embodiments, the methods of the invention comprise co-administering VX-222 and VX-950 independently for a period from about 4 weeks to about 12 weeks (e.g., about 4, 6, 8 or 12 weeks), for a period from about 20 weeks to about 28 weeks (e.g., about 20, 24, or 28 weeks), or for a period of from about 8 weeks to about 24 weeks (e.g., about 8, 12, 16 or 24 weeks). In one aspect of each of these embodiments, the administration of VX-222 and VX-950 independently (initial phase) is followed by administration of interferon and ribavirin (without VX-222 and VX-950) (secondary phase) for a period from about 4 weeks to about 36 weeks (e.g., from about 8 weeks to about 36 weeks, from about 8 weeks to about 24 weeks, from about 4 weeks to about 24 weeks). Specific exemplary regimens include: administering VX-222 and VX-950 independently for about 8 weeks followed by administering interferon and ribavirin (without VX-222 and VX-950) for about 16 weeks for a total treatment regimen of about 24 weeks; administering VX-222 and VX-950 independently for about 12 weeks followed by administering interferon and ribavirin (without VX-222 and VX-950) for about 12 weeks for a total treatment regimen of about 24 weeks. In such regimens, optionally is provided administration of interferon and ribavirin for the phase of administration of VX-222 and VX-950.

In certain embodiments, VX-222, and optionally VX-950 or interferon, are administered independently for less than about 12 weeks.

In certain embodiments, VX-222, and optionally VX-950 or interferon, are administered independently for about 8-12 weeks.

In certain embodiments, VX-222, and optionally VX-950 or interferon, are administered independently for about 10 weeks.

In certain embodiments, VX-222, and optionally VX-950 or interferon, are administered independently for less than about 10 weeks.

In certain embodiments, VX-222, and optionally VX-950 or interferon, are administered independently for about 2 weeks.

In other embodiments, VX-222, and optionally VX-950 or interferon, are administered independently for less than about 8 weeks (or about 8 weeks), less than about 6 weeks (or about 6 weeks), or less than about 4 weeks (or about 4 weeks).

In certain embodiments, VX-222 and VX-950 are co-administered for about 12 weeks (initial phase), optionally followed by interferon and ribavirin independently for about 12 weeks (secondary phase).

In certain embodiments, VX-222, VX-950 and interferon are co-administered for about 12 weeks, optionally followed by interferon and ribavirin independently for about 12 weeks (secondary phase).

In certain embodiments, VX-222, VX-950, interferon and ribavirin are co-administered for about 12 weeks, optionally followed by interferon and ribavirin independently for about 12 weeks (secondary phase).

In certain embodiments, VX-222, interferon and ribavirin are co-administered for about 12 weeks (initial phase), optionally followed by interferon and ribavirin independently for about 12 weeks (secondary phase).

In certain embodiments, VX-222 and VX-950 are co-administered for about 12 weeks (initial phase), optionally followed by interferon and ribavirin for about 12 weeks (secondary phase).

In certain embodiments, VX-222, VX-950, interferon, and ribavirin are co-administered for about 12 weeks (initial phase), optionally followed by interferon and ribavirin for about 12 weeks (secondary phase).

In certain embodiments, VX-222, VX-950, interferon and ribavirin are co-administered for about 12 weeks (initial phase), optionally followed by interferon and ribavirin for about 36 weeks (secondary phase).

In some embodiments, any of the initial phases described above can be conducted for less than 12 weeks and the secondary phases can be conducted for about 12 weeks. Alternatively, the initial phase can be conducted for about 12 weeks and the secondary phase can be conducted for about 24 weeks. In still other aspects, the initial phase can be conducted for about 8 weeks, and the secondary phase can be conducted for about 36 weeks. In still other aspects, the initial phase can be conducted for about 4 weeks, and the secondary phase can be conducted for about 36 weeks.

In some embodiments, any of the initial phases described above can be conducted for about 8 weeks and the secondary phases can be conducted for about 16 weeks. Alternatively, the initial phase can be conducted for about 8 weeks and the secondary phase can be conducted for about 40 weeks. In still other aspects, the initial phase can be conducted for about 8 weeks and the secondary phase can be conducted for about 40 weeks.

In some embodiments, any of the initial and secondary phases described above can be switched with each other, for example, to administer interferon (optionally with ribavirin) in the initial phase, and administer VX-222 (optionally with VX-950, or with VX-950, interferon, and ribavirin) in the secondary phase.

In some embodiments, the methods of the invention described above employ response-guided criteria in evaluating short-duration treatment regimens of about 12 weeks or about 24 weeks of total treatment. In these embodiments, patients who achieve undetectable HCV RNA (less than 10 IC/mL) at weeks 2 and 8 of treatment are randomized to either stop all treatment at 12 weeks or to receive an additional 12 weeks of peg-IFN (pegylated interferon) and RBV (ribavirin) therapy, for a total of about 24 weeks of treatment; and patients who do not achieve undetectable HCV RNA at weeks 2 and 8 receive an additional 12 week of Peg-IFN and RBV (without VX-222 or without VX-222 and VX-950) therapy for a total of 24 weeks of treatment.

In some embodiments, the methods of the invention described above employ response-guided criteria in evaluating short-duration treatment regimens of about 12 weeks, about 24 weeks or about 36 weeks of total treatment. In these embodiments, patients who achieve undetectable HCV RNA (less than 10 IC/mL) at weeks 2 and 8 of treatment will stop their assigned treatment at week 12. Patients who do not achieve undetectable HCV RNA at weeks 2 and 8 receive either an additional 12 weeks of Peg-IFN and RBV (without VX-222 or without VX-222 and VX-950) therapy for a total of 24 weeks of treatment, or an additional 24 weeks of Peg-IFN and RBV (without VX-222 or without VX-222 and VX-950) therapy for a total of 36 weeks of treatment. In some particular embodiments, the methods employ VX-950 and VX-222 (without Peg-IFN and RBV), such as VX-950 at 1125 mg twice per day and VX-222 at 100 mg or 400 mg twice per day, and patients who achieve undetectable HCV RNA (less than 10 IC/mL) at weeks 2 and 8 of treatment will stop their assigned treatment at week 12; and patients who do not achieve undetectable HCV RNA at weeks 2 and 8 receive an additional 24 weeks of Peg-IFN and RBV (without VX-222 or without VX-222 and VX-950) therapy for a total of 36 weeks of treatment. In some particular embodiments, the methods employ VX-950, VX-222, Peg-IFN and RBV (such as VX-950 at 1125 mg twice per day and VX-222 at 100 mg or 400 mg twice per day, Peg-IFN at 180 mcg once weekly, and RBV at 800 mg-1200 mg twice per day (e.g., 1000 mg for patients weighing less than 75 kb or 1200 mg for patients weighing more than or equal to 75 kg)), and patients who achieve undetectable HCV RNA (less than 10 IC/mL) at weeks 2 and 8 of treatment will stop their assigned treatment at week 12; and patients who do not achieve undetectable HCV RNA at weeks 2 and 8 receive an additional 12 weeks of Peg-IFN and RBV (without VX-222 or without VX-222 and VX-950) therapy for a total of 24 weeks of treatment.

As would be realized by skilled practitioners, if a method of this invention is being used to treat a patient prophylactically, and that patient becomes infected with Hepatitis C virus, the method may then treat the infection. Therefore, one embodiment of this invention provides methods for treating or preventing a Hepatitis C infection in a patient.

In addition to treating patients infected with Hepatitis C, the methods of this invention may be used to prevent a patient from becoming infected with Hepatitis C. Accordingly, one embodiment of this invention provides a method for preventing a Hepatitis C virus infection in a patient comprising administering to the patient VX-222 optionally with any additional HCV drugs, such as VX-950; interferon; interferon and ribavirin; VX-950, interferon, and ribavirin, as described above.

The methods of this invention may also involve administration of another component comprising an additional agent selected from an immunomodulatory agent; an antiviral agent; an inhibitor of HCV protease (other than VX-222, or VX-950); an inhibitor of another target in the HCV life cycle (other than NS3/4A protease); an inhibitor of internal ribosome entry, a broad-spectrum viral inhibitor; or a cytochrome P-450 inhibitor; or combinations thereof. The additional agent is also selected from an inhibitor of viral cellular entry.

Accordingly, in some embodiments, the additional agent is another anti-viral agent, preferably an anti-HCV agent (other than VX-222 or VX-950). Such anti-viral agents include, but are not limited to, immunomodulatory agents, such as α-, β-, and γ-interferons or thymosin, pegylated derivatized interferon-α compounds, and thymosin; other anti-viral agents, such as ribavirin, amantadine, and telbivudine; other inhibitors of hepatitis C proteases (NS2-NS3 inhibitors and NS3-NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds described in U.S. Pat. Nos. 5,807,876, 6,498,178, 6,344,465, and 6,054,472; and PCT publications WO 97/40028, WO 98/40381, and WO 00/56331; and mycophenolic acid and derivatives thereof, and including, but not limited to, VX-497, VX-148, and VX-944); or any of their combinations.

Other agents (e.g., non-immunomodulatory or immunomodulatory compounds) may be used in combination with a compound of this invention include, but are not limited to, those specified in WO 02/18369, which is incorporated herein by reference (see, e.g., page 273, lines 9-22 and page 274, line 4 to page 276, line 11 this disclosure being specifically incorporated herein by reference).

Still other agents include those described in various published U.S. patent applications. These publications provide additional teachings of compounds and methods that could be used in combination with VX-950 in the methods of this invention, particularly for the treatment of hepatitis. It is contemplated that any such methods and compositions may be used in combination with the methods and compositions of the present invention. For brevity, the disclosures from those publications are referred to with reference to the publication number but it should be noted that the disclosure of the compounds in particular is specifically incorporated herein by reference. Examples of such publications include U.S. Patent Application Publication Nos. US 20040058982, US 20050192212, US 20050080005, US 20050062522, US 20050020503, US 20040229818, US 20040229817, US 20040224900, US 20040186125, US 20040171626, US 20040110747, US 20040072788, US 20040067901, US 20030191067, US 20030187018, US 20030186895, US 20030181363, US 20020147160, US 20040082574, US 20050192212, US 20050187192, US 20050187165, US 20050049220, and US 20050222236.

Still other agents include, but are not limited to, Albuferon™ (albumin-Interferon alpha) available from Human Genome Sciences; PEG-INTRON® (peginterferon alfa-2b, available from Schering Corporation, Kenilworth, N.J.); INTRON-A®, (VIRAFERON®, interferon alfa-2b available from Schering Corporation, Kenilworth, N.J.); ribavirin (1-beta-D-ribofuranosyl-1H-1,2,4-triazole-3-carboxamide, available from ICN Pharmaceuticals, Inc., Costa Mesa, Calif.; described in the Merck Index, entry 8365, Twelfth Edition); REBETROL® (Schering Corporation, Kenilworth, N.J.); COPEGUS® (Hoffmann-La Roche, Nutley, N.J.); PEGASYS® (peginterferon alfa-2a available Hoffmann-La Roche, Nutley, N.J.); ROFERON® (recombinant interferon alfa-2a available from Hoffmann-La Roche, Nutley, N.J.); BEREFOR® (interferon alfa 2 available from Boehringer Ingelheim Pharmaceutical, Inc., Ridgefield, Conn.); SUMIFERON® (a purified blend of natural alpha interferons such as Sumiferon available from Sumitomo, Japan); WELLFERON° (interferon alpha n1 available from Glaxo Wellcome Ltd., Great Britain); ALFERON® (a mixture of natural alpha interferons made by Interferon Sciences, and available from Purdue Frederick Co., CT); α-interferon; natural alpha interferon 2a; natural alpha interferon 2b; pegylated alpha interferon 2a or 2b; consensus alpha interferon (Amgen, Inc., Newbury Park, Calif.); REBETRON® (Schering Plough, Interferon-alpha 2B+Ribavirin); pegylated interferon alpha (Reddy, K. R. et al., “Efficacy and Safety of Pegylated (40-kd) Interferon alpha-2a Compared with Interferon alpha-2a in Noncirrhotic Patients with Chronic Hepatitis C,” Hepatology, 33, 433-438 (2001); consensus interferon (INFERGEN®) (Kao, J. H., et al., “Efficacy of Consensus Interferon in the Treatment of Chronic Hepatitis,” J. Gastroenterol. Hepatol., 15, 1418-1423 (2000); lymphoblastoid or “natural” interferon; interferon tau (Clayette, P. et al., “IFN-tau, A New Interferon Type I with Antiretroviral activity” Pathol. Biol. (Paris) 47, 553-559 (1999); interleukin-2 (Davis, G. L. et al., “Future Options for the Management of Hepatitis C.” Seminars in Liver Disease, 19, 103-112 (1999); Interleukin-6 (Davis et al., “Future Options for the Management of Hepatitis C,” Seminars in Liver Disease, 19, 103-112 (1999); interleukin-12 (Davis, G. L. et al., “Future Options for the Management of Hepatitis C.” Seminars in Liver Disease, 19, 103-112 (1999); and compounds that enhance the development of type 1 helper T cell response (Davis et al., “Future Options for the Management of Hepatitis C,” Seminars in Liver Disease, 19, 103-112 (1999)). Also included are compounds that stimulate the synthesis of interferon in cells (Tazulakhova, E. B. et al., “Russian Experience in Screening, analysis, and Clinical Application of Novel Interferon Inducers” J. Interferon Cytokine Res., 21 65-73) including, but are not limited to, double stranded RNA, alone or in combination with tobramycin, and Imiquimod (3M Pharmaceuticals; Sauder, D. N. “Immunomodulatory and Pharmacologic Properties of Imiquimod,” J. Am. Acad. Dermatol., 43 S6-11 (2000). See also, WO 02/18369, particularly page 272, line 15 to page 273, line 8, this disclosure being specifically incorporated herein by reference.

Suitable examples of cytochrome P450 monooxygenase (“CYP”) inhibitors include, but are not limited to, ritonavir (WO 94/14436), ketoconazole, troleandomycin, 4-methyl pyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methylpyrazole, cyclosporin, and clomethiazole.

One embodiment of this invention provides a method for co-administering an inhibitor of CYP3A4.

Suitable examples of interferon that can be employed in the invention include Albuferon™ (albumin-Interferon alpha) available from Human Genome Sciences; PEG-INTRON® (peginterferon alfa-2b, available from Schering Corporation, Kenilworth, N.J.); INTRON-A®, (VIRAFERON®, interferon alfa-2b available from Schering Corporation, Kenilworth, N.J.); PEGASYS® (peginterferon alfa-2a available Hoffmann-La Roche, Nutley, N.J.); ROFERON® (recombinant interferon alfa-2a available from Hoffmann-La Roche, Nutley, N.J.); BEREFOR® (interferon alfa 2 available from Boehringer Ingelheim Pharmaceutical, Inc., Ridgefield, Conn.); SUMIFERON® (a purified blend of natural alpha interferons such as Sumiferon available from Sumitomo, Japan); WELLFERON® (interferon alpha n1 available from Glaxo Wellcome Ltd., Great Britain); ALFERON® (a mixture of natural alpha interferons made by Interferon Sciences, and available from Purdue Frederick Co., CT); alpha-interferon; natural alpha interferon 2a; natural alpha interferon 2b; pegylated alpha interferon 2a or 2b; consensus alpha interferon (Amgen, Inc., Newbury Park, Calif.); REBETRON® (Schering Plough, Interferon-alpha 2B+Ribavirin); pegylated interferon alpha (Reddy, K. R. et al. “Efficacy and Safety of Pegylated (40-kd) Interferon alpha-2a Compared with Interferon alpha-2a in Noncirrhotic Patients with Chronic Hepatitis C, Hepatology, 33, pp. 433-438 (2001)); consensus interferon (INFERGEN®) (Kao, J. H., et al., “Efficacy of Consensus Interferon in the Treatment of Chronic Hepatitis,” J. Gastroenterol. Hepatol. 15, pp. 1418-1423 (2000); lymphoblastoid or “natural” interferon; interferon tau (Clayette, P. et al., “IFN-tau, A New Interferon Type I with Antiretroviral activity,” Pathol. Biol. (Paris) 47, pp. 553-559 (1999)); and Omega Duros® delivering omega interferon via implantable Duros® (Intarcia Therapeutics, Inc., Mountain View, Calif.).

In some embodiments, the methods of the invention employ co-administering an interferon with or without ribavirin. Specifically, the interferon is a pegylated interferon (Peg-IFN). More specifically, the pegylated interferon is a pegylated interferon alpha, such as pegylated interferon alpha-2a or pegylated interferon alpha-2b.

In general, VX-222 and any additional HCV drugs (such as VX-950, interferon, and ribavirin) can independently be administered separately or together. Generally, VX-222 and VX-950 may independently be administered orally, parenterally, sublingually, by inhalation spray, topically, rectally, nasally, buccally, vaginally or via an implanted reservoir. Interferon is not typically administered orally, although orally administered forms are in development. Nevertheless, nothing herein limits the methods or combinations of this invention to any specific dosage forms or regime. As recognized by skilled practitioners, dosages of interferon are typically measured in IU (e.g., about 4 million IU to about 12 million IU). Interferon may also be dosed by micrograms. For example, a standard dose of Peg-Intron® is about 1.0-1.5 μg/kg/wk and of Pegasys® is about 180 μg/wk. Ribavirin is typically administered orally, and tablet forms of ribavirin are currently commercially available. General standard, daily dose of ribavirin tablets (e.g., about 200 mg tablets) is about 800 mg to about 1200 mg. For example, ribavirn tablets are administered at about 1000 mg for subjects weighing less than 75 kg, or at about 1200 mg for subjects weighing more than or equal to 75 kg. Nevertheless, nothing herein limits the methods or combinations of this invention to any specific dosage forms or regime. Typically, ribavirin can be dosed according to the dosage regimens described in its commercial product labels.

In a certain embodiment, VX-222 and VX-950 (when it is employed) are each independently administered orally or intravenously. In another certain embodiment, VX-222 and VX-950 (when it is employed) are each independently administered orally.

In some embodiments, the additional therapeutic agent is a cytochrome P-450 inhibitor. For CYP inhibitors, the dosage levels of between about 0.001 to about 200 mg/kg body weight per day, would be typical. More typical would be dosage levels of between about 0.1 to about 50 mg/kg or about 1.1 to about 25 mg/kg per day.

In some embodiments, the additional therapeutic agent is ritonavir. For specific dosage forms of ritonavir, see U.S. Pat. No. 6,037,157, and the documents cited therein: U.S. Pat. No. 5,484,801, U.S. patent application Ser. No. 08/402,690, and PCT Publications Nos. WO 95/07696 and WO 95/09614.

Generally in the invention, “administration” or “co-administration” of one or more therapeutic agents (including VX-950, interferon and ribavirin, and any combination thereof) includes administering each active therapeutic agent in the same dosage form or in different dosage forms. When administered in different dosage forms, the active therapeutic agent may be administered at different times, simultaneously, or in any time period around administration of the other dosage forms. Separate dosage forms may be administered in any order. That is, any dosage forms may be administered prior to, together with, or following the other dosage forms.

Generally, various dosage forms, formulation types and administration frequencies, and combinations thereof can be employed in the invention. Any suitable dosage form and formulation type can be employed in the invention.

In some embodiments, the methods of the invention comprises administering VX-222 in a dosage form containing about 100 mg of VX-222 (e.g., two capsules of 50 mg of VX-222) and the dosage form is administered once a day, two times per day, e.g., once every 12 hours (i.e., q12h), or three times per day, e.g., once every 8 hours (i.e., q8h). In some embodiments, the methods of the invention comprises administering VX-222 in a dosage form containing about 400 mg of VX-222 (e.g., two capsules of 200 mg of VX-222) and the dosage form is administered once a day, two times per day, e.g., once every 12 hours (i.e., q12h), or three times per day, e.g., once every 8 hours (i.e., q8h). In some embodiments, the methods of the invention comprises administering VX-222 in a dosage form containing about 750 mg of VX-222 (e.g., three capsules of 200 mg of VX-222 and three capsules of 50 mg of VX-222) and the dosage form is administered once a day, two times per day, e.g., once every 12 hours (i.e., q12h), or three times per day, e.g., once every 8 hours (i.e., q8h). In some embodiments, the methods of the invention comprises administering VX-222 in a dosage form containing about 1,500 mg of VX-222 (e.g., seven capsules of 200 mg of VX-222 and one capsule of 50 mg of VX-222) and the dosage form is administered once a day.

In one aspect of any one of the aforementioned methods of the invention, an oral dose of a composition comprising VX-950 is administered to a patient in need thereof, wherein said dose provides to the patient an average plasma concentration (C_{avg}) of VX-950 of at least about 750 ng/mL after the administration. In some specific embodiments, the C_{avg} of VX-950 is about 1000 ng/mL or about 1250 ng/mL. In some specific embodiments, said dose essentially contains about 750 mg of VX-950. In some specific embodiments, the C_{avg} is obtained or attained within 3 hours (e.g., 2 hours or 1 hour) after administration of VX-950. In some specific embodiments, the C_{avg} of VX-950 is maintained over about 24 hours (e.g., 5 weeks or 12 weeks). In another aspect, the oral dose provides to the patient a trough plasma VX-950 level minimum of about 750 ng/mL over the 24-hour period. In some specific embodiments, the dosage form is administered to maintain a trough plasma VX-950 level minimum of about 800 ng/mL (e.g., about 900 ng/mL or about 1000 ng/mL) over the 24 hour period. In yet another aspect, with the oral dose, a therapeutically effective plasma concentration is obtained and a certain trough level is maintained, wherein the trough VX-950 plasma level is maintained at a minimum of about 750, 800, 900, or 1000 ng/mL over a 24 hour period. In certain specific embodiments, trough levels of VX-950 are about 750, 800, 900, 1000 ng/mL to about 1500 ng/mL (e.g. 1000 to about 1500). In certain specific embodiments, trough levels of VX-950 are about 750, 800, 900, 1000 ng/mL to about 2500 ng/mL (particularly 1000 to about 2500). Also provided is a dosage form for delivering VX-950 to a human, wherein the dosage form comprises VX-950, said dosage form when administered at least once during a 24 hour period maintains a trough plasma VX-950 level that is at least about 750 ng/mL, 800 ng/mL, 900 ng/mL, or 1000 ng/mL over the 24 hour period to about 2500 ng/mL (e.g., 1000 ng/mL to about 2500 ng/mL, or 1000 ng/mL to about 1500 ng/mL) over the 24 hour period. In another aspect, the oral dose provides to the patient a mean AUC(_{0-24 hr}) of VX-950 in a range of about 30,000 hr*ng/mL to about 120,000 hr*ng/mL over a 24-hour period. In certain specific embodiments, the AUC(_{0-24 hr}) of VX-950 is in a range of about 50,000 hr*ng/mL to about 120,000 hr*ng/mL. In certain specific embodiments, the AUC(_{0-24 hr}) of VX-950 is in a range of about 60,000 hr*ng/mL to about 100,000 hr*ng/mL. In certain specific embodiments, the AUC(_{0-24 hr}) of VX-950 is in a range of about 60,000 hr*ng/mL to about 90,000 hr*ng/mL. Other specific dosage regimens of VX-950 disclosed in WO 2008/144072 and WO 2005/25517 can also be employed in the invention.

In one aspect of any one of the aforementioned methods of the invention, an oral dose of a composition comprising VX-222 is provided to a patient in need thereof, wherein said dose provides to the patient a mean maximum plasma concentration (Cₘₐₓ) of VX-222 of at least about 750 ng/mL after the administration. In some specific embodiments, the Cₘₐₓ of VX-222 is at least about 1,000 ng/mL. In some specific embodiments, the Cₘₐₓ of VX-222 in a range of about 750 ng/mL to about 15,000 ng/mL. In some specific embodiments, the Cₘₐₓ of VX-222 is in a range of about 1,000 ng/mL to about 15,000 ng/mL. In some specific embodiments, the Cₘₐₓ of VX-222 is in a range of about 3,000 ng/mL to about 15,000 ng/mL. In some specific embodiments, the Cₘₐₓ of VX-222 is in a range of about 3,000 ng/mL to about 12,000 ng/mL. In another aspect, an oral dose of a composition comprising VX-222 is provided to a patient in need thereof, wherein said dose provides to the patient a mean AUC(_{0-24 hr}) of VX-222 in a range of about 5,000 hr*ng/mL to about 150,000 hr*ng/mL over a 24-hour period. In some specific embodiments, the AUC₍₀₋₂₄₋ₕᵣ₎ of VX-222 is in a range of about 5,000 hr*ng/mL to about 125,000 hr*ng/mL. In some specific embodiments, the AUC(₀₋₂₄₋ₕᵣ) of VX-222 is in a range of about 20,000 hr*ng/mL to about 100,000 hr*ng/mL. In some specific embodiments, the AUC(₀₋₂₄₋ₕᵣ) of VX-222 is in a range of about 20,000 hr*ng/mL to about 80,000 hr*ng/mL.

VX-222 and any additional agent, may be formulated in separate dosage forms. Alternatively, to decrease the number of dosage forms administered to a patient, VX-222 and any additional agent, may be formulated together in any combination. Any separate dosage forms may be administered at the same time or different times. It should be understood that dosage forms should be administered within a time period such that the biological effects were advantageous.

For example, the amounts of each of VX-222 and VX-950 according to the invention can be administered in a single dosage form or in more than one dosage form. If in separate dosage forms, each dosage form is administered about simultaneously.

If pharmaceutically acceptable salts are employed in the invention as active therapeutic agents, those salts are typically derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentane-propionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups may be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

In the invention, as desired, modification of therapeutic agent(s) can also be employed by, for example, appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

Typically, one or more therapeutic agents, including VX-222 and VX-950, employed in the invention are included in pharmaceutical compositions, though the therapeutic agent(s) may be administered alone. A “pharmaceutical composition” means a composition comprising a therapeutic agent disclosed herein, and at least one component selected from the group comprising pharmaceutically acceptable carriers, diluents, coatings, adjuvants, excipients, or vehicles, such as preserving agents, fillers, disintegrating agents, wetting agents, emulsifying agents, emulsion stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage forms. The compositions may be presented in the form of tablets, pills, granules, powders, aqueous solutions or suspensions, injectable solutions, elixirs or syrups.

Exemplary suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Exemplary antibacterial and antifungal agents for the prevention of the action of microorganisms include parabens, chlorobutanol, phenol, sorbic acid, and the like. Exemplary isotonic agents include sugars, sodium chloride and the like. Exemplary adsorption delaying agents to prolong absorption include aluminum monosterate and gelatin. Exemplary adsorption promoting agents to enhance absorption include dimethyl sulphoxide and related analogs. Exemplary carriers, diluents, solvents, vehicles, solubilizing agents, emulsifiers and emulsion stabilizers, include water, chloroform, sucrose, ethanol, isopropyl alcohol, ethyl carbonate, ethyl acetate, benzyl alcohol, tetrahydrofurfuryl alcohol, benzyl benzoate, polyols, propylene glycol, 1,3-butylene glycol, glycerol, polyethylene glycols, dimethylformamide, Tween 60, Span & commat; 80, cetostearyl alcohol, myristyl alcohol, glyceryl mono-stearate and sodium lauryl sulfate, fatty acid esters of sorbitan, vegetable oils (such as cottonseed oil, groundnut oil, corn germ oil, olive oil, castor oil and sesame oil) and injectable organic esters such as ethyl oleate, and the like, or suitable mixtures of these substances. Exemplary excipients include lactose, milk sugar, sodium citrate, calcium carbonate, dicalcium phosphate. Exemplary disintegrating agents include starch, alginic acids and certain complex silicates. Exemplary lubricants include magnesium stearate, sodium lauryl sulphate, talc, as well as high molecular weight polyethylene glycols.

The choice of material in the pharmaceutical composition other than the therapeutic agent is generally determined in accordance with the chemical properties of the therapeutic agent, such as solubility, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets.

The pharmaceutical compositions may be presented in assorted forms such as tablets, pills, granules, powders, aqueous solutions or suspensions, injectable solutions, elixirs or syrups.

“Liquid dosage form” means the dose of the therapeutic agent to be administered to the patient is in liquid form, for example, pharmaceutically acceptable emulsions, solutions, suspensions, syrups and elixirs. In addition to the active compounds, the liquid dosage forms may contain inert diluents commonly used in the art, such solvents, solubilizing agents and emulsifiers.

Solid compositions may also be employed as fillers in soft and hard-filled gelatin capsules using such excipients as lactose or milk sugar as well as high molecular weight polyethylene glycols, and the like.

When aqueous suspensions are used they can contain emulsifying agents or agents which facilitate suspension.

The oily phase of the emulsion pharmaceutical composition may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier that acts as a stabilizer. It is also preferred to include both an oil and a fat. Together, the emulsifier(s) with or without stabilizer(s) make up the emulsifying wax, and the way together with the oil and fat make up the emulsifying ointment base which forms the oily dispersed phase of the cream formulations.

If desired, the aqueous phase of the cream base may include, for example, a least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas.

The choice of suitable oils or fats for a formulation is based on achieving the desired cosmetic properties. Thus the cream should preferably be a non-greasy, non-staining and washable product with suitable consistency to avoid leakage from tubes or other containers.

Straight or branched chain, mono- or di-basic alkyl esters such as di-isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

Generally, a therapeutic agent/pharmaceutical compositions disclosed herein may be administered in a suitable formulation to humans and animals by topical or systemic administration, including oral, inhalational, rectal, nasal, buccal, sublingual, vaginal, colonic, parenteral (including subcutaneous, intramuscular, intravenous, intradermal, intrathecal and epidural), intracisternal and intraperitoneal. It will be appreciated that the preferred route may vary with for example the condition of the recipient.

“Pharmaceutically acceptable dosage forms” refers to dosage forms of a therapeutic agent (including VX-950) disclosed herein, and includes, for example, tablets, powders, elixirs, syrups, liquid preparations, including suspensions, sprays, inhalants tablets, lozenges, emulsions, solutions, granules, capsules and suppositories, as well as liquid preparations for injections, including liposome preparations. Techniques and formulations generally may be found in Remington's Pharmaceutical Sciences, Mack Publishing Co., Easton, Pa., latest edition.

“Formulations suitable for oral administration” may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tables may be prepared by compressing in a suitable machine the active ingredient in a free-flowing form such as a powder or granules, optionally mixed with a binder, lubricant, inert diluent, preservative, surface active or dispersing agent. Moulded tablets may be made by moulding in a suitable machine a mixture of the powdered compounds moistened with an inert liquid diluent. The tablets may optionally be coated or scored and may be formulated so as to provide slow or controlled release of the active ingredient therein.

Solid compositions for rectal administration include suppositories formulated in accordance with known methods and containing at least one compound of the invention.

If desired, and for more effective distribution, a therapeutic agent disclosed herein can be microencapsulated in, or attached to, a slow release or targeted delivery systems such as a biocompatible, biodegradable polymer matrices (e.g., poly (d, l-lactide co-glycolide)), liposomes, and microspheres and subcutaneously or intramuscularly injected by a technique called subcutaneous or intramuscular depot to provide continuous slow release of the compound (s) for a period of 2 weeks or longer. The therapeutic agent may be sterilized, for example, by filtration through a bacteria retaining filter, or by incorporating sterilizing agents in the form of sterile solid compositions which can be dissolved in sterile water, or some other sterile injectable medium immediately before use.

“Formulations suitable for nasal or inhalational administration” means formulations which are in a form suitable to be administered nasally or by inhalation to a patient. The formulation may contain a carrier, in a powder form, having a particle size for example in the range 1 to 500 microns (including particle sizes in a range between 20 and 500 microns in increments of 5 microns such as 30 microns, 35 microns, etc.) Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents. Inhalational therapy is readily administered by metered dose inhalers.

“Formulations suitable for oral administration” means formulations which are in a form suitable to be administered orally to a patient. The formulations may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The therapeutic agent may also be presented as a bolus, electuary or paste.

“Formulations suitable for parenteral administration” means formulations that are in a form suitable to be administered parenterally to a patient. The formulations are sterile and include emulsions, suspensions, aqueous and non-aqueous injection solutions, which may contain suspending agents and thickening agents and anti-oxidants, buffers, bacteriostats and solutes which render the formulation isotonic, and have a suitably adjusted pH, with the blood of the intended recipient.

“Formulations suitable for rectal or vaginal administrations” means formulations that are in a form suitable to be administered rectally or vaginally to a patient. The formulation is preferably in the form of suppositories that can be prepared by mixing the compounds of this invention with suitable non-irritating excipients or carriers such as cocoa butter, polyethylene glycol or a suppository wax, which are solid at ordinary temperatures but liquid at body temperature and therefore, melt in the rectum or vaginal cavity and release the active component.

“Formulations suitable for systemic administration” means formulations that are in a form suitable to be administered systemically to a patient. The formulation is preferably administered by injection, including transmuscular, intravenous, intraperitoneal, and subcutaneous. For injection, the compounds of the invention are formulated in liquid solutions, preferably in physiologically compatible buffers such as Hank's solution or Ringer's solution. In addition, the compounds may be formulated in solid form and redissolved or suspended immediately prior to use. Lyophilized forms are also included. Systematic administration also can be by transmucosal or transdermal means, or the compounds can be administered orally. For transmucosal or transdermal administration, penetrants appropriate to the barrier to be permeated are used in the formulation. Such penetrants are generally known in the art, and include, for example, bile salts and fusidic acid derivatives for transmucosal administration. In addition, detergents may be used to facilitate permeation. Transmucosal administration may be through use of nasal sprays, for example, or suppositories. For oral administration, the compounds are formulated into conventional oral administration forms such as capsules, tablets, and tonics.

“Formulations suitable for topical administration” means formulations that are in a form suitable to be administered topically to a patient. The formulation may be presented as a topical ointment, salves, powders, sprays and inhalants, gels (water or alcohol based), creams, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. Formulations suitable for topical administration in the eye include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

“Solid dosage form” means the dosage form of a therapeutic agent disclosed herein is solid form, for example capsules, tablets, pills, powders, dragees or granules. In such solid dosage forms, the compound of the invention is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, (i) lubricants, as for example, talc, calcium stearate, magnesium stearate, solid polyethylene glycols, sodium lauryl (j) opacifying agents, (k) buffering agents, and agents which release the compound (s) of the invention in a certain part of the intestinal tract in a delayed manner.

The amount of active therapeutic agent(s) that may be combined with the carrier and/or excipient materials to produce a single dosage form will vary depending upon the host treated and the particular mode of administration. A typical preparation will contain from about 5% to about 95% active therapeutic agent (w/w). Preferably, such preparations contain from about 20% to about 80% therapeutic agent.

The formulations can be prepared in unit dosage form by any of the methods well known in the art of pharmacy. Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

The formulations may be presented in unit-dose or multi-dose containers, for example sealed ampoules and vials with elastomeric stoppers, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

The pharmaceutical compositions and dosage formulations disclosed herein are preferably for use in vivo. Nevertheless, this is not intended as a limitation to using of the pharmaceutical compositions and dosage formulations for any purpose. For example, a biological substance pre-treated with a pharmaceutical composition disclosed herein can also be employed in the invention. Such biological substances include, but are not limited to, blood and components thereof such as plasma, platelets, subpopulations of blood cells and the like; organs such as kidney, liver, heart, lung, etc; sperm and ova; bone marrow and components thereof, and other fluids to be infused into a patient such as saline, dextrose, etc.

It should also be understood that a specific dosage and treatment regimen for any particular patient will depend upon a variety of factors, including the activity of the specific compound employed, the age, body weight, general health, sex, diet, time of administration, rate of excretion, drug combination, the judgment of the treating physician and the severity of the particular disease being treated, prior treatment history, co-morbidities or concomitant medications, baseline viral load, race, duration of diseases, status of liver function and degree of liver fibrosis/cirrhosis, and the goal of therapy (eliminating circulating virus per-transplant or viral eradication). The amount of active ingredients will also depend upon the particular described compound and the presence or absence and the nature of the additional anti-viral agent in the composition.

According to the treatment regimens and dosage forms of this invention, co-therapy of VX-950 and interferon is effective to decrease the viral load in a sample or in a patient, wherein said virus encodes a NS3/4A serine protease necessary for the viral life cycle (or in an amount effective to carry out a method of this invention). Accordingly, the invention also provides a method for treating a patient infected with a virus characterized by a virally encoded NS3/4A serine protease that is necessary for the life cycle of the virus by administering to said patient VX-950 and interferon (and optionally one or more additional therapeutic agent) as described above.

In the invention, each active therapeutic agent employed in the invention, independently, can be administered to a patient with or without food. In some embodiments, VX-222 and/or any additional HCV drugs independently are administered with food. As used herein, the phrase “in combination with food” means that the active therapeutic agent(s) is administered within about 90 minutes of consumption of food, e.g., no more than about 90 minutes after food is eaten and no more than about 90 minutes prior to eating period. In some embodiments, the active therapeutic agent(s) is administered up to about 30 minutes before, or up to 30 minutes after consumption of food. Although not required, and any type of food (high fat or low fat) can be consumed, a high-fat meal may provide improved absorption as compared to lower fat meals. As used herein, “high fat” means food in which over about 30% of the calories are provided by fat. In a certain embodiment, the food has at least about 50 calories. In another certain embodiment, the food has at least about 100 calories. In yet another certain embodiment, the food has at least about 50-100 calories up to about 3,000 calories, up to about 2,000 calories, or up to about 1,000 calories. In yet another certain embodiment, the food includes at least about 30% of its total calories from fat.

Generally in the invention, treatment may completely eradicate the HCV viral infection or reduce the severity thereof, such that an effective therapeutic response is achieved. An effective therapeutic response may be, for example, one or both of a) achieving a sustained viral response; and b) achieving undetectable HCV RNA in the plasma by at least about 12 weeks (about 12 weeks or more). The term “undetectable” is as defined above.

In other embodiments, a method of this invention treats a patient infected with HCV such that the level of HCV RNA in the patient after the administration is at least about 2 log₁₀ (e.g., at least about 4 log₁₀) lower than that before the treatment.

In some embodiments, a relatively rapid drop in viral plasma concentration may be obtained by administering a loading dose to a patient. In one embodiment, the loading dose is about 1250 mg of VX-950.

In some embodiments, the method of this invention is able to achieve week 4 RVR and week 12 undetectable status.

Generally in the invention, a “patient” includes a mammal, particularly a human being.

In certain embodiments, a method of the invention provides treatment of a patient infected with genotype 1 Hepatitis C virus. It is generally believed that genotype 1 HCV infection is the most difficult strain of HCV to treat and the most prevalent strain in the United States.

Advantageously, both HCV treatment naïve and previously treated patients benefit from the methods of this invention. For the avoidance of doubt, patients that may be treated according to the methods of this invention include those where HCV treatment has not been tried or has failed, including non-responding, rebound, relapse, and breakthrough patients. In certain embodiments, the methods of the present invention treat HCV treatment naïve patient. As used herein, an “HCV treatment naïve” patient means that the patient has no previous HCV treatment with a drug(s) approved, or seeking approval, by the U.S. Food and Drug Administration (FDA), or any other U.S. or international agency equivalent to the U.S. FDA.

The methods of the invention can be used as a chronic or acute therapy. As would be realized by skilled practitioners, if a method of this invention is being used to treat a patient prophylactically, and that patient becomes infected with Hepatitis C virus, the method may then treat the infection. Therefore, one embodiment of this invention provides methods for treating or preventing a Hepatitis C infection in a patient.

The assay for determined VX-222 and VX-950 concentrations in patient's plasma can be performed by methods well known in the art. See, e.g., Wasley, A. et al., Semin Liver Dis., 20:1-16, 2000; Alter, H. J. et al., Semin. Liver Dis., 20: 17-35, 2000; Brown, R. S. Jr. et al., Liver Transpl., 9: S10-S13, 2003; DeFrancesco, R. et al., Nature, 436(7053): 953-960, 2005; Bowen, D. G. et al., J. Hepatol., 42: 408-417, 2005; Hoofnagle, J. H., Hepatology, 36: S21-S29, 2002, Brown, R. S. Jr. et al., Nature, 436 (7053): 973-978, 2005; and Chisari, F. V., Nature, 436(7053): 930-932, 2005.

Administrations in connection with this invention can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Preferably, such preparations contain from about 20% to about 80% active compound.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

It should also be understood that a specific dosage and treatment regimen for any particular patient will depend upon a variety of factors, including the activity of the specific compound employed, the age, body weight, general health, sex, diet, time of administration, rate of excretion, drug combination, the judgment of the treating physician and the severity of the particular disease being treated, prior treatment history, co-morbidities or concomitant medications, baseline viral load, race, duration of diseases, status of liver function and degree of liver fibrosis/cirrhosis, and the goal of therapy (eliminating circulating virus per-transplant or viral eradication). The amount of active ingredients will also depend upon the particular described compound and the presence or absence and the nature of the additional anti-viral agent in the composition.

According to another embodiment, the invention provides a method for treating a patient infected with a virus characterized by a virally encoded NS3/4A serine protease that is necessary for the life cycle of the virus by administering to said patient a pharmaceutically acceptable composition of this invention. Preferably, the methods of this invention are used to treat a patient suffering from a HCV infection. Such treatment may completely eradicate the viral infection or reduce the severity thereof. Preferably, the patient is a mammal. More preferably, the patient is a human being.

The dosages herein are preferably for use in vivo. Nevertheless, this is not intended as a limitation to using of these amounts of, for example, VX-222 or VX-950 for any purpose. In yet another embodiment the present invention provides a method of pre-treating a biological substance intended for administration to a patient comprising the step of contacting said biological substance with a pharmaceutically acceptable composition comprising a compound of this invention. Such biological substances include, but are not limited to, blood and components thereof such as plasma, platelets, subpopulations of blood cells and the like; organs such as kidney, liver, heart, lung, etc; sperm and ova; bone marrow and components thereof, and other fluids to be infused into a patient such as saline, dextrose, etc.

This invention also provides a process for preparing a composition comprising VX-222, VX-950, and a pharmaceutically acceptable carrier, adjuvant, or vehicle, comprising the step of combining VX-222, VX-950, and the pharmaceutically acceptable carrier, adjuvant, or vehicle, wherein the dosage of each of VX-222 and VX-950 in the composition is independently in accordance with any embodiment of this invention. An alternative embodiment of this invention provides a process wherein the composition comprises one or more additional agent as described herein.

Pharmaceutical compositions may also be prescribed to the patient in “patient packs” containing the whole course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in traditional prescriptions. The inclusion of a package insert has been shown to improve patient compliance with the physician's instructions.

It will be understood that the administration of the combination of the invention by means of a single patient pack, or patient packs of each formulation, containing within a package insert instructing the patient to the correct use of the invention is a desirable additional feature of this invention.

According to a further aspect of the invention is a pack including VX-222 (in dosages according to this invention) and an information insert containing directions on the use of the combination of the invention. Any composition, dosage form, therapeutic regimen or other embodiment of this invention may be presented in a pharmaceutical pack. In an alternative embodiment of this invention, the pharmaceutical pack further comprises one or more of additional agent as described herein. The additional agent or agents may be provided in the same pack or in separate packs.

Another aspect of this involves a packaged kit for a patient to use in the treatment of HCV infection or in the prevention of HCV infection (or for use in another method of this invention), comprising: a single or a plurality of pharmaceutical formulation of each pharmaceutical component; a container housing the pharmaceutical formulation(s) during storage and prior to administration; and instructions for carrying out drug administration in a manner effective to treat or prevent HCV infection.

Accordingly, this invention provides kits for the simultaneous or sequential administration of a dose of VX-222 (and optionally an additional agent). Typically, such a kit will comprise, e.g. a composition of each compound and optional additional agent(s) in a pharmaceutically acceptable carrier (and in one or in a plurality of pharmaceutical formulations) and written instructions for the simultaneous or sequential administration.

In another embodiment, a packaged kit is provided that contains one or more dosage forms for self administration; a container means, preferably sealed, for housing the dosage forms during storage and prior to use; and instructions for a patient to carry out drug administration. The instructions will typically be written instructions on a package insert, a label, and/or on other components of the kit, and the dosage form or forms are as described herein. Each dosage form may be individually housed, as in a sheet of a metal foil-plastic laminate with each dosage form isolated from the others in individual cells or bubbles, or the dosage forms may be housed in a single container, as in a plastic bottle. The present kits will also typically include means for packaging the individual kit components, i.e., the dosage forms, the container means, and the written instructions for use. Such packaging means may take the form of a cardboard or paper box, a plastic or foil pouch, etc.

A kit according to this invention could embody any aspect of this invention such as any composition, dosage form, therapeutic regimen, or pharmaceutical pack.

The packs and kits according to this invention optionally comprise a plurality of compositions or dosage forms. Accordingly, included within this invention would be packs and kits containing one composition or more than one composition.

Although certain exemplary embodiments are depicted and described below, it will be appreciated that compounds of this invention can be prepared according to the methods described generally above using appropriate starting materials generally available to one of ordinary skill in the art.

VX-222 can be prepared in general by methods known to those skilled in the art (see, e.g., WO 2002/100851 and WO 2008/058393). Any suitable formulations known in the art can be employed in the invention. For example, formulations described in WO 2002/100851 and WO 2008/058393 can be employed in the invention. One specific example that can be employed in the invention includes: free acid form of VX-222; Avicel PH 101; Lactose Monhydrate; Poloxamer 188; Sodium Lauryl Sulfate; Providone K29/32; Avicel PH 102; Lactose Monohydrate; Crosscarmellose Sodium; Magnesium Stearate. A specific formulation that can be used in the invention is exemplified in Example 5.

One embodiment of the invention is a formulation of VX-222 comprising free acid form of VX-222 (a compound represented by Structural Formula (I)); Avicel PH 101; Lactose Monhydrate; Poloxamer (e.g., Polxamer 188); Sodium Lauryl Sulfate; Providone K29/32; Avicel PH 102; Lactose Monohydrate; Crosscarmellose Sodium; and Magnesium Stearate. In a specific embodiment, the formulation includes about 45-60 wt % of the free acid form of VX-222; about 5-20 wt % of Avicel PH 101; about 10-20% of Lactose monohydrate; about 1-10 wt % of Poloxamer (e.g., Polxamer 188); about 1-5 wt % of Sodium Lauryl Sulfate; about 1-10 wt % of Providone (e.g., Providone K29/32); about 1-10 wt % of Avicel PH 102; about 1-10 wt % of Lactose Monohydrate; about 1-10 wt % of Crosscarmellose Sodium; and about 0.1-5 wt % of Magnesium Stearate. The formulations described in Example 5 are also included in the invention.

VX-950 can be prepared in general by methods known to those skilled in the art (see, e.g., WO 02/18369). Any suitable formulations known in the art can be used in the invention. For example, formulations described in WO 2005/123075, WO 2007/109604, WO 2007/109605 and WO 2008/080167 can be employed in the invention. A specific formulation that can be used in the invention is exemplified in Example 4. Other specific examples include:

| | |
|---|---|
| VX-950 | 49.5 wt % |
| HPMC 40 cp | 49.5 wt % |
| SLS | 1 wt % |
| VX-950 | 49.5 wt % |
| HPC | 49.5 wt % |
| SLS | 1 wt % |
| VX-950 | 49.5 wt % |
| PVP K30 | 49.5 wt % |
| SLS | 1 wt % |

VX-950 Solid Dispersion

| % (w/w) | Ingredient | |
|---|---|---|
| 49.5 | VX-950 | Spray-dried |
| 49.5 | PVP K29/32 | from a MeCl₂ |
| 1 | SLS | solution |

wherein HPMC (Hydroxypropyl Methylcellulose 60SH 50 cP (Biddle Sawyer or Shin-Etsu Metolose, HPMC60SH50) (Hypromellose Acetate Succinate, HG grade, Shin-Etsu Chemical Co.) HPC (hydroxypropyl cellulose), PVP (polyvinylpyrrolidone) and SLS (Sodium Lauryl Sulfate) are as described in WO 2005/123075. In certain embodiments, the solid dispersion shown above can be suspended in a 1% HPMC, 0.002% simethicone solution (1 wt % HPMC, 0.002 wt % simethicone and 99 wt % water). Additional examples include 1:1 VX950: PVPK30, 1 wt % SLS (Refreshed Tox.); Niro-49 wt % HPMCAS/1 wt % SLS/1 wt % SDBS/49% VX-950; 40.5 wt % PVP-VA/10 wt % ETPGS/49.5 wt % VX-950; 40.5 wt % HPMC/10 wt % ETPGS/49.5 wt % VX-950; 49 wt % VX950, 49 wt % HPMCAS, 1 wt % SLS, 1 wt % SDBS; and 49 wt % VX950, 16 wt % HPPh, 33 wt % HPC, 1 wt % SLS, wt % SDBS, wherein PVPK30 (Polyvinyl Pyrrolidone K30), SDBS (sodium dodecyl benzene sulfonate), HPMCAS (Hydroxypropyl Methylcellulose Acetate Succinate), Vitamin ETPGS, PVP (polyvinylpyrrolidone) and SLS (Sodium Lauryl Sulfate), and details of the preparation of these formulations can be found in WO 2005/123075. Additional examples include those described in WO 2007/109604:

a solid dispersion comprising 55 wt % VX-950, 24.4 wt % HPMCAS-HG (Hydroxypropyl Methylcellulose Acetate Succinate, JPE (Biddle Sawyer or Shin-Etsu HPMCAS-HG grade), 19.6 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 55 wt % VX-950, 14.7 wt % HPMCAS-HG (Hydroxypropyl Methylcellulose Acetate Succinate, JPE (Biddle Sawyer or Shin-Etsu HPMCAS-HG grade), 29.3 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 60 wt % VX-950, 24.4 wt % HPMCAS-HG (Hydroxypropyl Methylcellulose Acetate Succinate, JPE (Biddle Sawyer or Shin-Etsu HPMCAS-HG grade), 14.6 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 65 wt % VX-950, 17 wt % HPMCAS-HG (Hydroxypropyl Methylcellulose Acetate Succinate, JPE (Biddle Sawyer or Shin-Etsu HPMCAS-HG grade), 17 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 70 wt % VX-950, 9.7 wt % HPMCAS-HG (Hydroxypropyl Methylcellulose Acetate Succinate, JPE (Biddle Sawyer or Shin-Etsu HPMCAS-HG grade), 19.3 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 60 wt % VX-950, 39 wt % HPMCAS-HG (Hydroxypropyl Methylcellulose Acetate Succinate, JPE (Biddle Sawyer or Shin-Etsu HPMCAS-HG grade), and 1 wt % Sodium Lauryl Sulfate (SLS);

a solid dispersion comprising 49.5 wt % VX-950, 24.5 wt % HPMCAS-HG (Hydroxypropyl Methylcellulose Acetate Succinate, JPE (Biddle Sawyer or Shin-Etsu HPMCAS-HG grade), 24.5 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 83 wt % VX-950, 8 wt % HPMCAS-HG (Hydroxypropyl Methylcellulose Acetate Succinate, JPE (Biddle Sawyer or Shin-Etsu HPMCAS-HG grade), 8 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 49.5 wt % VX-950, 24.5 wt % HPMCAS-HG (Hydroxypropyl Methylcellulose Acetate Succinate, JPE (Biddle Sawyer or Shin-Etsu HPMCAS-HG grade), 24.5 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 70 wt % VX-950, 14.5 wt % HPMCAS-HG (Hydroxypropyl Methylcellulose Acetate Succinate, JPE (Biddle Sawyer or Shin-Etsu HPMCAS-HG grade), 14.5 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 65 wt % VX-950, 14.6 wt % HPMCAS-HG (Hydroxypropyl Methylcellulose Acetate Succinate, JPE (Biddle Sawyer or Shin-Etsu HPMCAS-HG grade), 19.4 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 65 wt % VX-950, 9.7 wt % HPMCAS-HG (Hydroxypropyl Methylcellulose Acetate Succinate, JPE (Biddle Sawyer or Shin-Etsu HPMCAS-HG grade), 24.3 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 60 wt % VX-950, 19.5 wt % HPMCAS-HG (Hydroxypropyl Methylcellulose Acetate Succinate, JPE (Biddle Sawyer or Shin-Etsu HPMCAS-HG grade), 19.5 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 60 wt % VX-950, 14.6 wt % HPMCAS-HG (Hydroxypropyl Methylcellulose Acetate Succinate, JPE (Biddle Sawyer or Shin-Etsu HPMCAS-HG grade), 24.4 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 70 wt % VX-950, 9.7 wt % HPMCAS-HG (Hydroxypropyl Methylcellulose Acetate Succinate, JPE (Biddle Sawyer or Shin-Etsu HPMCAS-HG grade), 19.3 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 49.5 wt % VX-950, 24.5 wt % HPMCAS-HG (Hydroxypropyl Methylcellulose Acetate Succinate, JPE (Biddle Sawyer or Shin-Etsu HPMCAS-HG grade), 24.5 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 83 wt % VX-950, 8 wt % HPMCAS-HG (Hydroxypropyl Methylcellulose Acetate Succinate, JPE (Biddle Sawyer or Shin-Etsu HPMCAS-HG grade), 8 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 49.5 wt % VX-950, 49.5 wt % HPMCAS-HG (Hydroxypropyl Methylcellulose Acetate Succinate, JPE (Biddle Sawyer or Shin-Etsu HPMCAS-HG grade), and 1 wt % Sodium Lauryl Sulfate (SLS);

a solid dispersion comprising 83 wt % VX-950, 16 wt % HPMCAS-HG (Hydroxypropyl Methylcellulose Acetate Succinate, JPE (Biddle Sawyer or Shin-Etsu HPMCAS-HG grade), and 1 wt % Sodium Lauryl Sulfate (SLS);

a solid dispersion comprising 82.44 wt % VX-950, 15.89 wt % HPMCAS-HG (Hydroxypropyl Methylcellulose Acetate Succinate, JPE (Biddle Sawyer or Shin-Etsu HPMCAS-HG grade), and 1.67 wt % Sodium Lauryl Sulfate (SLS);

a solid dispersion comprising 49.5 wt % VX-950, 24.75 wt % HPMCAS-HG (Hydroxypropyl Methylcellulose Acetate Succinate, JPE (Biddle Sawyer or Shin-Etsu HPMCAS-HG grade), 24.75 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS).

a solid dispersion comprising 60 wt % VX-950, 24.6 wt % HPMCAS-HG (Hydroxypropyl Methylcellulose Acetate Succinate, JPE (Biddle Sawyer or Shin-Etsu HPMCAS-HG grade), 14.4 wt % HPMC-60SH (Hydroxypropyl Methylcellulose 60SH 50 cP (Biddle Sawyer or Shin-Etsu Metolose, HPMC60SH50), and 1 wt % Sodium Lauryl Sulfate (SLS);

a solid dispersion comprising 60 wt % VX-950, 39 wt % HPMCAS-HG (Hydroxypropyl Methylcellulose Acetate Succinate, JPE (Biddle Sawyer or Shin-Etsu HPMCAS-HG grade), and 1 wt % Sodium Lauryl Sulfate (SLS); and

a solid dispersion comprising 49.5 wt % VX-950, 49.5 wt % HPMCAS-HG (Hydroxypropyl Methylcellulose Acetate Succinate, JPE (Biddle Sawyer or Shin-Etsu HPMCAS-HG grade), and 1 wt % Sodium Lauryl Sulfate (SLS).

Details of the preparation of these solid dispersions are described in WO 2007/109604. Additional specific examples include tablet formulations containing a spray dried dispersion of VX-950, which are described in WO 2007/109604:

| | mg per | |
|---|---|---|
| Component | Tablet | Percent |
| Roller compaction blend | | |
| VX950 Spray Dried Dispersion1 | 505.1 | 74.9 |
| Pharmatose DCL 22 (Lactose, USP/NF, PhEur, | 37.5 | 5.6 |
| Ac-Di-Sol (cross carmellose sodium, NF, PhEur, | 24.0 | 3.6 |
| Extragranular addition | | 0.0 |
| Avicel pH 113 | 33.7 | 5.0 |
| Vitamin E TPGS (NF) | 24.0 | 3.6 |
| Ac-Di-Sol (cross carmellose sodium, NF, PhEur, | 16.0 | 2.4 |
| Cabosil M-5 (colloidal silicon dioxide, NF, PhEur) | 8.0 | 1.2 |
| Sodium Stearyl fumarate (NF, PhEur, JP) | 26.0 | 3.9 |
| Total Formulation weight | 674.3 | 100.0 |

Additional specific examples include tablet formulations described in WO2008/080167:

VX950 SD Tableting Experiment Design
(Potency: 250 mg VX950)

| Trial # | Vit E type | Vit E type |
|---|---|---|
| A | VitE-TPGS (24 mg) | Granulated VitE on excipients |
| C | VitE-Acetate (48 mg) | Used as is |
| E | Vit E-TPGS(24 mg) | Vit E Spray Congealed |
| F | Vit E-TPGS (24 mg) | Granulated Vit E onto VX950 |

Trial#A Formulation

| | | Wt/Tablet | |
|---|---|---|---|
| Item | Ingredients | (mg) | wt % |
| | Physical mixture | | |
| 1 | Solid Dispersion | 339.9 | 66.32 |
| | (73.55%VX950/26.45%HPMCAS) | | |
| 2 | PHARMATOSE ® DCL 22 (Lactose) | 37.5 | 7.32 |
| 3 | AC-DI-SOL ® (Cross carmellose sodium) | 24.0 | 4.68 |
| 4 | Sodium Stearyl Fumarate | 1.6 | 0.32 |
| 5 | SLS | 3.4 | 0.66 |
| 6 | AVICEL ® pH 113 (Microcrystalline cellulose) | 33.7 | 6.58 |
| 7 | Vitamin E TPGS (granulated on excipients) | 24.0 | 4.68 |
| 8 | AC-DI-SOL ® (Cross carmellose sodium) | 16.0 | 3.12 |
| 9 | Cabosil M-5 (Colloidal silicon dioxide) | 8.0 | 1.56 |
| 10 | Sodium Stearyl Fumarate | 24.4 | 4.76 |
| | Total | 512.5 | 100 |

Note:
VX 950 SD Lot 02
Potency: 250 mg VX950

Trial# C Formulation

| | | Wt/Tablet | |
|---|---|---|---|
| Item | Ingredients | (mg) | wt % |
| | Physical mixture | | |
| 1 | Solid Dispersion | 339.9 | 63.36 |
| | (73.55%VX950/26.45%HPMCAS) | | |
| 2 | PHARMATOSE ® DCL 22 (Lactose) | 37.5 | 6.99 |
| 3 | AC-DI-SOL ® (Cross carmellose sodium) | 24.0 | 4.47 |
| 4 | Sodium Stearyl Fumarate | 1.6 | 0.30 |
| 5 | SLS | 3.4 | 0.63 |
| 6 | AVICEL ® pH 113 (Microcrystalline | 33.7 | 6.28 |
| | cellulose) | | |
| 7 | Vitamin E-Acetate | 48.0 | 8.95 |
| 8 | AC-DI-SOL ® (Cross carmellose sodium) | 16.0 | 2.98 |
| 9 | Cabosil M-5 (Colloidal silicon dioxide) | 8.0 | 1.49 |
| 10 | Sodium Stearyl Fumarate | 24.4 | 4.54 |
| | Total | 536.5 | 100 |

Trial# E Formulation

| | | Wt/Tablet | |
|---|---|---|---|
| Item | Ingredients | (mg) | wt % |
| | Physical mixture | | |
| 1 | Solid Dispersion | 339.9 | 66.32 |
| | (73.55%VX950/26.45%HPMCAS) | | |
| 2 | PHARMATOSE ® DCL 22 (Lactose) | 37.5 | 7.32 |
| 3 | AC-DI-SOL ® (Cross carmellose sodium) | 24.0 | 4.68 |
| 4 | Sodium Stearyl Fumarate | 1.6 | 0.32 |
| 5 | SLS | 3.4 | 0.66 |
| 6 | AVICEL ® pH 113 (Microcrystalline | 33.7 | 6.58 |
| | cellulose) | | |
| 7 | Vitamin E Spray Congealed | 24.0 | 4.68 |
| 8 | AC-DI-SOL ® (Cross carmellose sodium) | 16.0 | 3.12 |
| 9 | Cabosil M-5 (Colloidal silicon dioxide) | 8.0 | 1.56 |
| 10 | Sodium Stearyl Fumarate | 24.4 | 4.76 |
| | Total | 512.5 | 100 |

Note:
VX 950 SD Lot 02
Potency: 250 mg VX950

Trial# F Formulation

| | | Wt/Tablet | |
|---|---|---|---|
| Item | Ingredients | (mg) | wt % |
| 1 | Solid Dispersion | 339.9 | 66.32 |
| | (73.55%VX950/26.45%HPMCAS) | | |
| 2 | Vitamin E granulated onto dispersion | 24.0 | 4.68 |
| 3 | PHARMATOSE ® DCL 22 (Lactose) | 37.5 | 7.32 |
| 4 | AC-DI-SOL ® (Cross carmellose sodium) | 24.0 | 4.68 |
| 5 | Sodium Stearyl Fumarate | 1.6 | 0.32 |
| 6 | SLS | 3.4 | 0.66 |
| 7 | AVICEL ® pH 113 (Microcrystalline | 33.7 | 6.58 |
| | cellulose) | | |
| 8 | AC-DI-SOL ® (Cross carmellose sodium) | 16.0 | 3.12 |
| 9 | Cabosil M-5 (Colloidal silicon dioxide) | 8.0 | 1.56 |
| 10 | Sodium Stearyl Fumarate | 24.4 | 4.76 |
| | Total | 512.5 | 100 |

Note:
VX 950 SD Lot 02
Potency: 250 mg VX950

All cited documents are incorporated herein by reference.

In order that this invention be more fully understood, the following preparative and testing examples are set forth. These examples are for the purpose of illustration only and are not to be construed as limiting the scope of the invention in any way.

### EXEMPLIFICATION

In the following examples, VX-222 refers to Compound 1 and VX-950 refers to Compound 2.

### Example 1

### HCV Replicon Cell Assay Protocol

Cells containing hepatitis C virus (HCV) replicon were maintained in DMEM containing 10% fetal bovine serum (FBS), 0.25 mg per ml of G418, with appropriate supplements (media A).

On day 1, replicon cell monolayer was treated with a trypsin:EDTA mixture, removed, and then media A was diluted into a final concentration of 100,000 cells per ml with 10,000 cells in 100 μl were plated into each well of a 96-well tissue culture plate, and cultured overnight in a tissue culture incubator at 37° C.

On day 2, compounds (in 100% DMSO) were serially diluted into DMEM containing 2% FBS, 0.5% DMSO, with appropriate supplements (media B). The final concentration of DMSO was maintained at 0.5% throughout the dilution series.

Media on the replicon cell monolayer was removed, and then media B containing various concentrations of compounds was added. Media B without any compound was added to other wells as no compound controls.

Cells were incubated with compound or 0.5% DMSO in media B for 48 hours in a tissue culture incubator at 37° C. At the end of the 48-hour incubation, the media was removed, and the replicon cell monolayer was washed once with PBS and stored at −80° C. prior to RNA extraction.

Culture plates with treated replicon cell monolayers were thawed, and a fixed amount of another RNA virus, such as Bovine Viral Diarrhea Virus (BVDV) was added to cells in each well. RNA extraction reagents (such as reagents from RNeasy kits) were added to the cells immediately to avoid degradation of RNA. Total RNA was extracted according the instruction of manufacturer with modification to improve extraction efficiency and consistency. Finally, total cellular RNA, including HCV replicon RNA, was eluted and stored at −80° C. until further processing.

A Taqman real-time RT-PCR quantification assay was set up with two sets of specific primers and probe. One was for HCV and the other was for BVDV. Total RNA extractants from treated HCV replicon cells was added to the PCR reactions for quantification of both HCV and BVDV RNA in the same PCR well. Experimental failure was flagged and rejected based on the level of BVDV RNA in each well. The level of HCV RNA in each well was calculated according to a standard curve run in the same PCR plate. The percentage of inhibition or decrease of HCV RNA level due to compound treatment was calculated using the DMSO or no compound control as 0% of inhibition. The IC50 (concentration at which 50% inhibition of HCV RNA level is observed) was calculated from the titration curve of any given compound.

VX-950 demonstrated significant activity in the replicon assay. VX-950 was shown to have an IC₅₀ of 240 ng/ml and IC₉₀ of 476 ng/ml.

### Example 2

### HCV Ki Assay Protocol

HPLC Microbore Method for Separation of 5AB Substrate and Products

Substrate: NH₂-Glu-Asp-Val-Val-(alpha)Abu-Cys-Ser-Met-Ser-Tyr-COOH SEQ ID NO: 1.

A stock solution of 20 mM 5AB (or concentration of your choice) was made in DMSO w/0.2M DTT. This was stored in aliquots at −20 C.

Buffer: 50 mM HEPES, pH 7.8; 20% glycerol; 100 mM NaCl

Total assay volume was 100 μL

| | X1 (μL) | conc. in assay |
|---|---|---|
| Buffer | 86.5 | See above |
| 5 mM KK4A | 0.5 | 25 μM |
| 1M DTT | 0.5 | 5 mM |
| DMSO or inhibitor | 2.5 | 2.5% v/v |
| 50 μM tNS3 | 0.05 | 25 nM |
| 250 μM 5AB (initiate) | 20 | 25 μM |

The buffer, KK4A, DTT, and tNS3 were combined; distributed 78 μL each into wells of 96 well plate. This was incubated at 30° C. for bout 5-10 minutes. 2.5 μL of appropriate concentration of test compound was dissolved in DMSO (DMSO only for control) and added to each well. This was incubated at room temperature for 15 min. The reaction was initiated by addition of 20 μL of 250 μM 5AB substrate (25 μM concentration is equivalent or slightly lower than the Km for 5AB). After incubating the reaction mixture for 20 min at 30° C., the reaction was terminated by addition of 25 μL of 10% TFA, and the mixture was transferred 120 μL aliquots to HPLC vials for analysis. SMSY product was separated from substrate and KK4A by the following method:

### Microbore Separation Method:

### Instrumentation: Agilent 1100

### Degasser G1322A

Binary pump G1312A

### Autosampler G1313A

Column thermostated chamber G1316A
Diode array detector G1315A

### Column:

Phenomenex Jupiter; 5 micron C18; 300 angstroms; 150×2 mm; P/O 00F-4053-B0
Column thermostat: 40 C
Injection volume: 100 μL
Solvent A=HPLC grade water+0.1% TFA
Solvent B=HPLC grade acetonitrile+0.1% TFA

| Time (min) | % B | Flow (ml/min) | Max press. |
|---|---|---|---|
| 0 | 5 | 0.2 | 400 |
| 12 | 60 | 0.2 | 400 |
| 13 | 100 | 0.2 | 400 |
| 16 | 100 | 0.2 | 400 |
| 17 | 5 | 0.2 | 400 |

Stop time: 17 min; Post-run time: 10 min.

### Example 3

VX-950 was examined in a randomized, double-blind, placebo-controlled single-dose escalation study. 25 healthy male volunteers were enrolled. Each subject received multiple single doses of VX-950 at least 7 days apart, 3 doses of VX-950 at increasing dose levels and 1 dose of placebo.

Doses of 25 mg to 1250 mg were evaluated. A dose escalation scheme was used that combined dose doubling and modified Fibonacci to be aggressive in the lower dose range and conservative in the higher dose range.

VX-950 was well tolerated at all dose levels and no serious adverse events were reported during the study. There did not appear to be an increase in adverse events with increasing dose levels.

A pharmacokinetics analysis was performed using the statistical moment approach. Pharmacokinetic analysis showed that VX-950 was absorbed with a median tₘₐₓ of 3 hours. Less than 2% of VX-950 was eliminated unchanged in the urine, indicating that the drug is primarily eliminated via the metabolic route.

### Example 4

An oral dosage formulation was prepared as follows. VX-950 and povidone K29/32 were dissolved in methylene chloride, then sodium lauryl sulfate was added and dispersed in the solution to form a homogenous suspension. This suspension was spray-dried using an inlet temperature of 90° C. and an outlet temperature of 56° C., and the product was collected from the cyclone. The spray-dried dispersion was fluid-bed dried at 75° C. for 8 hours. The resultant powder was pre-measured into glass vials, and just prior to dosing was suspended in water (30 mL) for administration to the subjects. In connection with dosing, each vial was washed with 3 separate portions of water, with the total volume of water being 90 mL.

VX-950 Solid Dispersion

| % (w/w) | Ingredient | |
|---|---|---|
| 49.5 | VX-950 | Spray-dried from CH₂Cl₂ |
| 49.5 | PVP K29/32 | |
| 1 | SLS | |

### Example 5

Two different oral dosage formulations of VX-222 (here Compound 1) were prepared as follows.

200 mg VX-222 Capsule formulation

| Ingredients | Amounts (mg) | Percent | 10 (kg) |
|---|---|---|---|
| VX-222 (free form, | 200.00 | 52.00 | 5.20 |
| Form A) | | | |
| Avicel PH 101 | 42.3 | 11.00 | 1.10 |
| Lactose | 53.8 | 14.00 | 1.40 |
| Monohydrate | | | |
| Poloxamer 188 | 13.5 | 3.50 | 0.35 |
| Sodium Lauryl | 7.7 | 2.00 | 0.20 |
| Sulfate | | | |
| Povidone K29/32 | 19.2 | 5.00 | 0.50 |
| Avicel PH 102 | 11.5 | 3.00 | 0.30 |
| Lactose | 11.5 | 3.00 | 0.30 |
| Monohydrate | | | |
| Crosscarmellose | 21.2 | 5.50 | 0.55 |
| Sodium | | | |
| Magnesium Stearate | 3.8 | 1.00 | 0.10 |
| Total Formulation | 384.62 | 100.00 | 10.00 |
| Weight (mg) | | | |

Final Weight

| | | |
|---|---|---|
| Hard gelatin | 100 | 2.60 |
| Capsule white | | |
| opaque, size 0 | | |
| Total Weight | 484.62 | 12.60 |

50 mg VX-222 Capsule formulation

| Ingredients | Amounts (mg) | Percent | 10 (kg) |
|---|---|---|---|
| VX-222 (free form: | 50.00 | 11.00 | 1.10 |
| Form A) | | | |
| Avicel PH 101 | 63.64 | 14.00 | 1.40 |
| Lactose | 172.73 | 38.00 | 3.80 |
| Monohydrate | | | |
| Poloxamer 188 | 15.91 | 3.50 | 0.35 |
| Sodium Lauryl | 9.09 | 2.00 | 0.20 |
| Sulfate | | | |
| Povidone K29/32 | 22.73 | 5.00 | 0.50 |
| Avicel PH 102 | 36.36 | 8.00 | 0.80 |
| Lactose | 54.55 | 12.00 | 1.20 |
| Monohydrate | | | |
| Crosscarmellose | 25.00 | 5.50 | 0.55 |
| Sodium | | | |
| Magnesium Stearate | 4.55 | 1.00 | 0.10 |
| Total Formulation | 454.55 | 100.00 | 10.00 |
| Weight (mg) | | | |

Final Weight

| | | |
|---|---|---|
| Hard gelatin | 100 | 2.20 |
| Capsule white | | |
| opaque, size 0 | | |
| Total Weight | 554.55 | 12.20 |

Certain characteristics of Form A of VX-222 (here Compound 1) are described below:

| Form A | |
|---|---|
| DSC Endotherm (° C.) | |
| 188° C. | |
| XRPD Peaks | |
| Angle (2-Theta ± 0.2) | Intensity % |
| 6.9 | 100.0 |
| 16.6 | 53.3 |
| 21.7 | 31.6 |
| 8.6 | 31.3 |
| 11.6 | 26.2 |
| 19.4 | 23.8 |

The XRPD patterns were acquired at room temperature in reflection mode using a Bruker D8 Discover diffractometer (Asset Tag V012842) equipped with a sealed tube source and a Hi-Star area detector (Bruker AXS, Madison, Wis.). The X-Ray generator was operating at a voltage of 40 kV and a current of 35 mA. The powder sample was placed in an aluminum holder. Two frames were registered with an exposure time of 120 s each. The data were subsequently integrated over the range of 4°-40° 2θ with a step size of 0.02° and merged into one continuous pattern.

Form A of VX-222 can be prepared by following the steps described below:

Charge 10 g of VX-222 (Compound 1 as prepared as described in WO 2008/058393) to a reactor

Charge 20 g of Methanol and heat to 60 C to dissolve.

Cool to 10 C, wait for solids to form.

Filter the solids

Add 20 g of Acetone at 25 C

Stir for 1 hour

Filter the solids

Dry at 75 C for 12 hours.

### Example 6

### Pharmacokinetic Data from the Study VX-222-002: Combination Treatment with VX-222 and VX-950

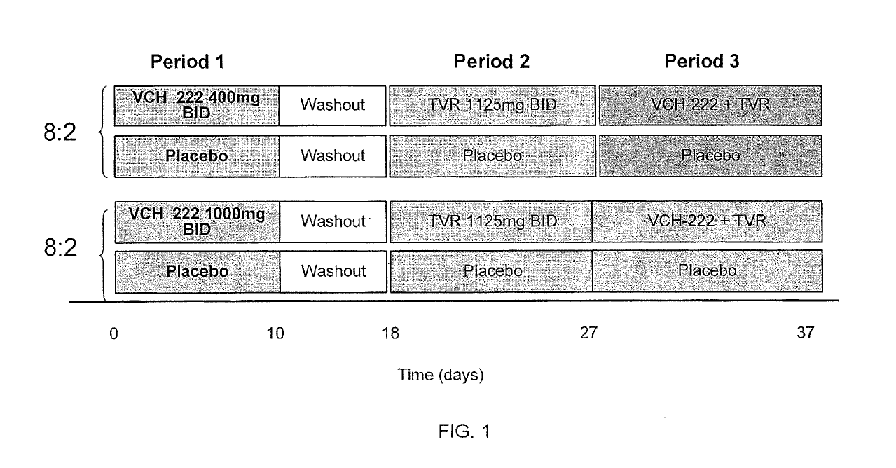

The formulations of Example 5 were used for VX-222. In the study, 20 healthy subjects were enrolled in Cohort 1 or Cohort 2 (10 subjects per cohort). Subjects in Cohort 1 and Cohort 2 completed all 3 periods of the study, Treatment Period 1, Treatment Period 2, and Treatment Period 3. In Treatment Period 1, subjects were administered VX-222 or VX-222 placebo for 10 days in the fed state. In Treatment Period 2, subjects were administered VX-950 or VX-950 placebo for 10 days in the fed state. In Treatment Period 3, subjects were simultaneously administered both VX-222 and VX-950 or both VX-222 placebo and VX-950 placebo for 10 days in the fed state. Treatment Period 1 and Treatment Period 2 were separated by a 7-day washout period because VX-222 was administered in Treatment Period 1 and VX-950 was administered in Treatment Period 2. Subjects in Cohort 1 were administered 400 mg VX-222 or VX-222 placebo every 12 hours (q12h) in Treatment Period 1 and Treatment Period 3. Subjects in Cohort 2 were administered 1,000 mg VX-222 or VX-222 placebo q12h in Treatment Period 1 and Treatment Period 3. Subjects in Cohort 1 and Cohort 2 are administered 1125 mg VX-950 or VX-950 placebo q12h in Period 2 and Period 3. Study design of this study is shown in FIG. 1:

Double-Blind, Placebo-Controlled, Parallel Group, Sequential, Dose Ascending
N=20 (10 per cohort (8:2))
Design:
   VCH-222 for 10 days (followed by washout period)
   VX-950 for 10 days
   VCH-222 and VX-950 for 10 days
   Follow-up—7+/−3 days
Doses:
   VX-950:
      Fixed dose of 1125 mg BID
   VCH-222:
      400 mg BID (Cohort 1)
      1000 mg BID (Cohort 2)
VX-950 and VCH-222 administered simultaneously with meal

Preliminary safety analyses indicated that in Treatment Period 3 there were no reported severe or SAEs. The majority of the reported adverse events were mild in severity and there were no untoward adverse event incidences or trends. Some adverse events, including diarrhea, decreased appetite, pruritus, epistaxis, and nasal congestion, were more common in Treatment Period 3 compared to Treatment Period 1 and Treatment Period 3.

Pharmacokinetic assessments were done as follows:

VCH-222 Plasma:
Period 1
   Day 1: 0 (predose), 0.5, 1, 1.5, 2, 4, 6, 8 and 12 hours post-dose
   Days: 3, 5, 7, 8 and 9: 0 (pre-dose)
   Day 10: 0 (predose), 0.5, 1, 1.5, 2, 4, 6, 8, 12, 24, 48 and 72 hours post-dose
Period 3
   Days: 31 and 33: 0 (pre-dose)
   Day 37: 0 (predose), 0.5, 1, 1.5, 2, 4, 6, 8, 12 and 24 hours post-dose

VX-950 (and metabolites) Plasma:
Period 2
   Days 22, 24 and 26: 0 (pre-dose)
   Day 27: 0 (pre-dose), 0.5, 1, 1.5, 2, 4, 6, 8 and 12 hours post-dose
Period 3
   Days: 31 and 33: 0 (pre-dose)
   Day 37: 0 (pre-dose), 0.5, 1, 1.5, 2, 4, 6, 8 and 12 hours post-dose

VCH-222 Urine:
Days 10 and 37: 0 to 4, 4 to 8, 8 to 12 and 12 to 24 (Day 10 only) hours post-dose

Table 1 below provides preliminary pharmacokinetic (PK) results of the study. As shown in the table, VX-222 plasma exposures were increased.

TABLE 1
PK Data of VX-222 (DDI)

| | | ^{a}Cmax | ^{a}AUC₀₋₁₂ | | | |
|---|---|---|---|---|---|---|
| Dose | | (ng/mL) | (ng*hr/mL) | ^{a}C₁₂ (ng/mL) | ^{b}Tmax (hr) | ^{c}T_{1/2} (hr) |
| 400 mg | Day 10 | 1773 (39%) | 8559 (47%) | 212 (71%) | 4 [0;4] | 2,7 (11%) |
| BID | Day 37 | 3954 (43%) | 29534 (46%) | 830 (54%) | 4 [4,6] | 5.4 (39%) |
| (N = 7/8) | | | | | | |
| 1000 mg | Day 10 | 4116 (33%) | 25842 (22%) | 589 (37%) | 4 [2;6] | 4 (29%) |
| BID | Day 37 | 8151 (51%) | 59892 (47%) | 1580 (79%0 | 4 [2;6] | 3.5 (15%) |
| (N = 8) | | | | | | |

^{a}Geometric Mean (CV %);
^{b}Arithmetic Mean (CV %);
^{c}Mean [Min; Max]

### Example 7

### Pharmacokinetic Data from the Study VX-222-102 in Part A: Treatment with VX-222

The formulations of Example 5 were used for VX-222. In Part A of Study 102 subjects were randomized to VX-222 or placebo in a 6:2 (VX-222: placebo) allocation ratio to either Cohort 1, Cohort 2, Cohort 3, or Cohort 4. Subjects enrolled in Cohort 1, Cohort 2, and Cohort 3 were administered 250 mg, 500 mf, or 750 mg VX-222 or placebo twice daily (b.i.d.) for 3 days, respectively. Subjects enrolled in Cohort 4 were administered 1,500 mg VX-222 or placebo once daily (qd) for 3 days. Standard of care treatment, Peg-IFN-alpha-2a and RBV was offered to the subjects at the end of dosing in Part A for up to 48 weeks, if judged appropriate by the physician.

### A. Preliminary Results

Preliminary Safety Analyses

Subjects with genotype 1 chronic hepatitis C infection were exposed to multiple doses of VX-222 or placebo at 250 mg (Cohort 1), 500 mg (Cohort 2), or 750 mg (Cohort 3) b.i.d. for 3 days. Preliminary safety analyses indicated that there were no reported severe or serious events. The majority of the reported adverse events were mild in severity and there were no untoward adverse incidences or trends.

Preliminary Pharmacokinetic (PK) Analyses

A summary of preliminary PK parameters from Cohort 1, Cohort 2 and Cohort 3 are presented in Table 2.

TABLE 2
PK Data of VX-222 in HCV Infected Subjects
Administered VX-222/Placebo for 3 Days

| | Dose | | | | | | | | | |
|---|---|---|---|---|---|---|---|---|---|---|
| | | | | | AUC_{0-12 hr} | | | | | |
| | | | Cₘₐₓ (ng/mL) | | (hr*ng/mL) | | C₁₂ (ng/mL) | | ^{c}T_{1/2} (hr) | |
| | tₘₐₓ (hr) | | Geometric Mean | | Geometric Mean | | Geometric Mean | | Arithmetic Mean | |
| | Median Range | | (CV %) | | (CV %) | | (CV %) | | (CV %) | |
| Dose | Day 1 | Day 3 | Day 1 | Day 3 | Day 1 | Day 3 | Day 1 | Day 3 | Day 1 | Day 3 |
| 250 mg | 6 | 4 | 1812 | 2959 | 11690 | 19490 | 858 | 728 | ND | 4.1 |
| N = 6 | [4-8] | [2-8] | (66) | (29) | (60) | (41) | (78) | (65) | | (22) |
| 500 mg | 3 | 2.5 | 4363 | 5044 | 22875 | 29848 | 767 | 858 | ND | 4 |
| N = 6 | [1.5-6] | [1-6] | (52) | (36) | (54) | (54) | (95) | (120) | | (32) |
| 750 mg | 3 | 2 | 6522 | 10288 | 43552 | 62952 | 1691 | 1677 | ND | 3.5 |
| N = 6 | [3,4] | [1-6] | (91) | (112) | (87) | (112) | (103) | (153) | | (26) |

^{a}Geometric Mean (CV %);
^{b}Arithmetic Mean (CV %);
^{c}Mean [Min; Max]

Preliminary HCV RNA Analyses

A summary of preliminary HCV RNA analyses from Cohort 1, Cohort 2, and Cohort 3 are provided in Table 3. The mean log HCV RNA decreases on Day 4 for subjects infected with genotype 1 HCV in Cohort 1, Cohort 2, and Cohort 3 were 3.1, 3.4, and 3.2, respectively. The mean log HCV RNA decrease on Day 4 for subjects infected with genotype 1 HCV in Cohort 4 where subjects administered 1,500 mg of VX-222 once a day was 3.6.

TABLE 3
Mena Viral Load Reduction at Day 4

| | 250 mg | 500 mg | 750 mg | 1,500 mg | |
|---|---|---|---|---|---|
| | b.i.d. (N = 6) | b.i.d. (N = 6) | b.i.d. (N = 6) | QD (N = 6) | Placebo (N = 8) |
| Mean (range) | −3.1 | −3.4 | −3.2 | −3.4 | −0.1 |
| | (−4.2; −2.0) | (−3.6; −3.2) | (−3.8; −2.3) | (−3.9; −3.1) | (−0.5; 0.3) |

### B. Additional Results

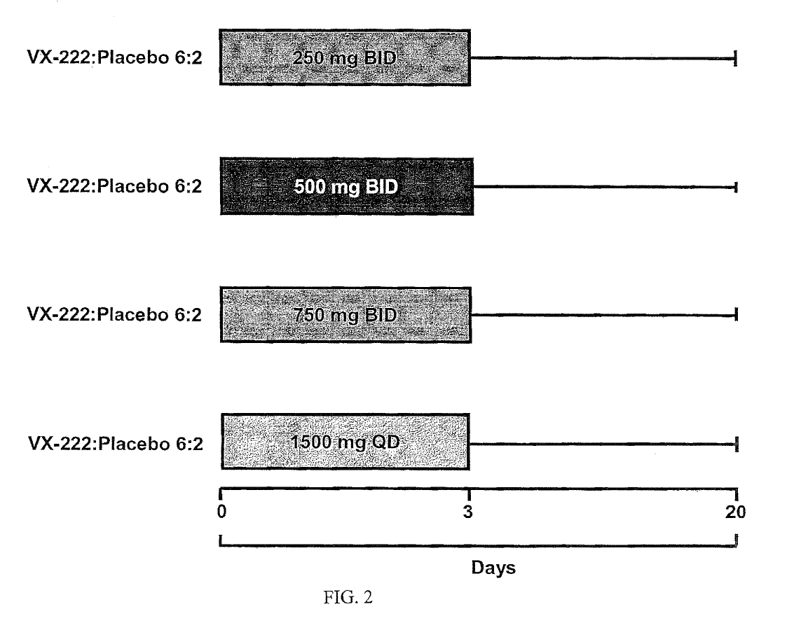

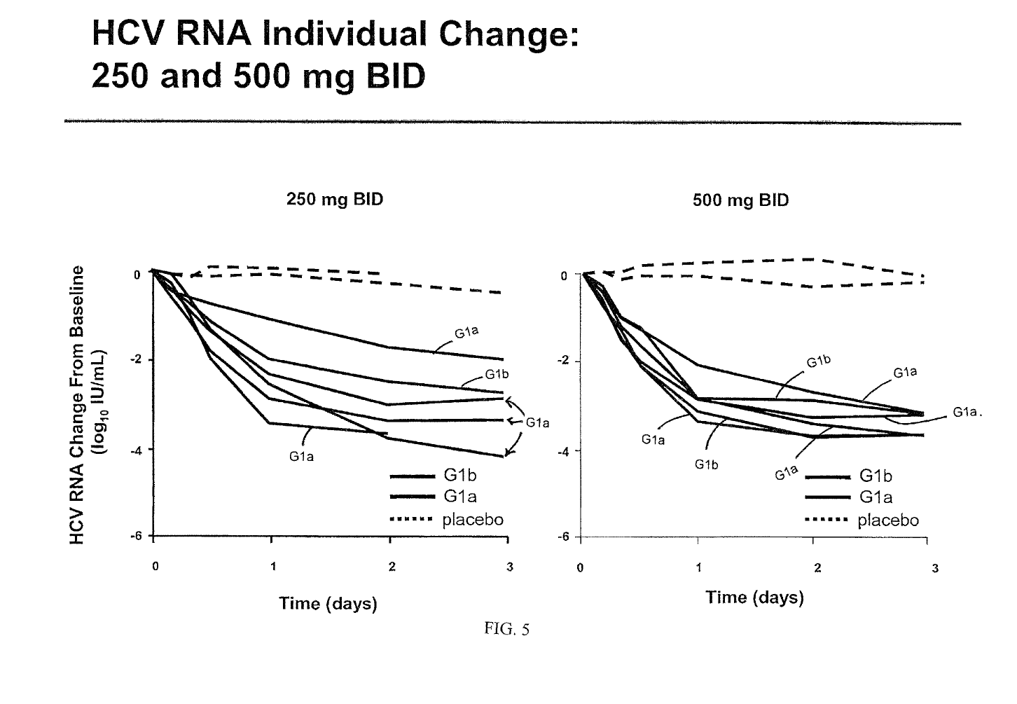

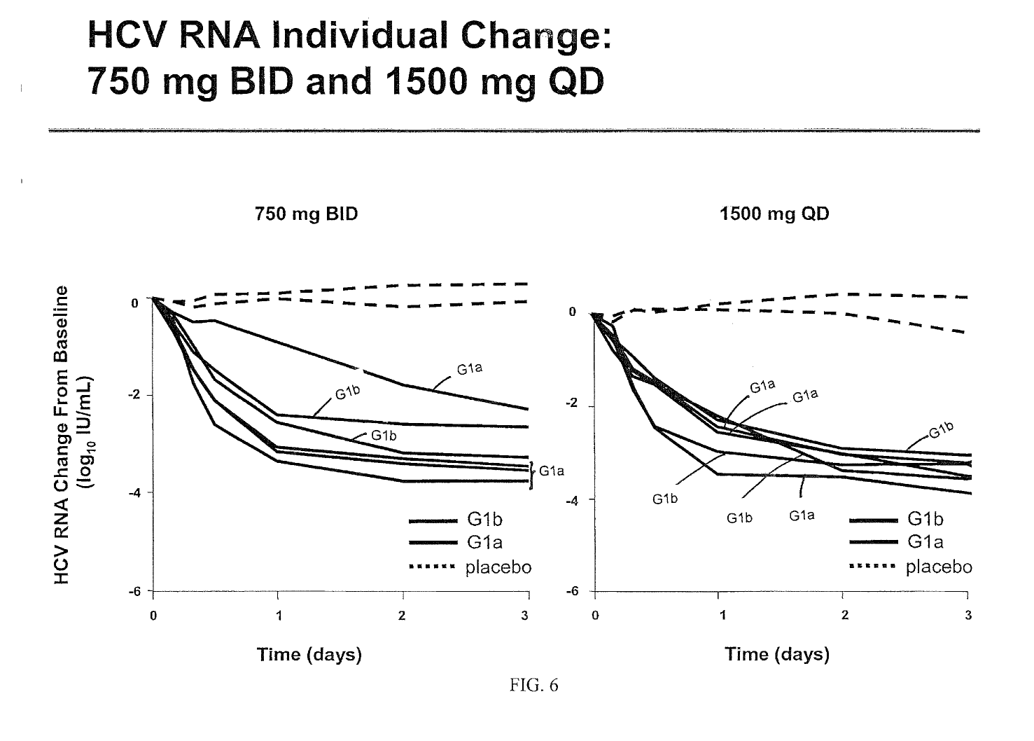

Additional results of Study VX-222-102 are described in FIGS. 2-8: Thirty-two treatment-naïve patients with chronic genotype 1 HCV infection were enrolled in the trial, including six patients in each dose group who received 250 mg of VX-222 BID, 500 mg of VX-222 BID, 750 mg of VX-222 BID, and 1,500 mg of VX-222 QD. Two patients received placebo in each of the four dosing groups, for a total of eight patients who received placebo. Part A of the trial was conducted at 10 centers in the United States, Canada and Argentina. Of the patients enrolled in the trial, 24 patients had genotype 1a HCV infection and eight patients had genotype 1b HCV infection. Six of the patients enrolled in the trial were African American, 25 were Caucasian and one was American Indian/Alaskan.

### Study Design a & Population

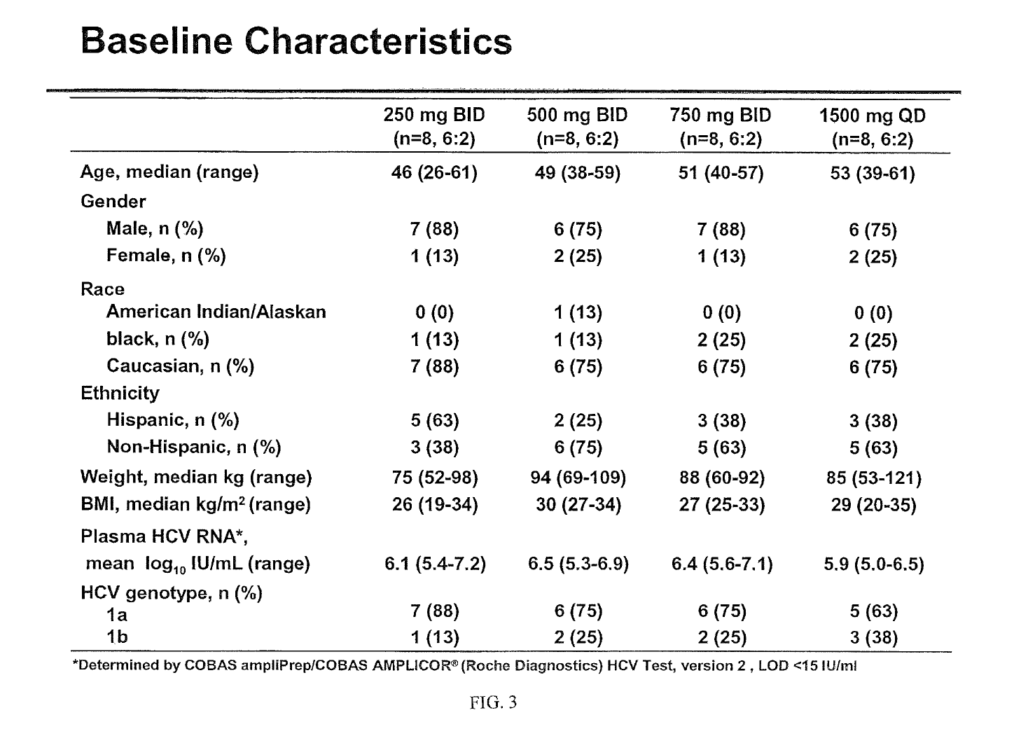

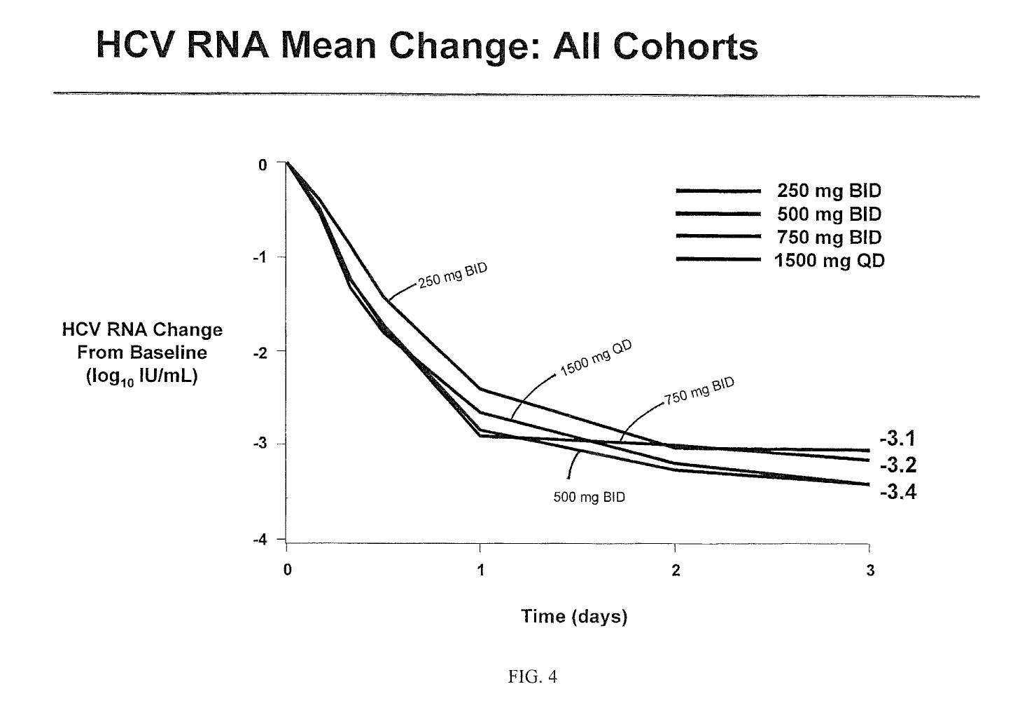

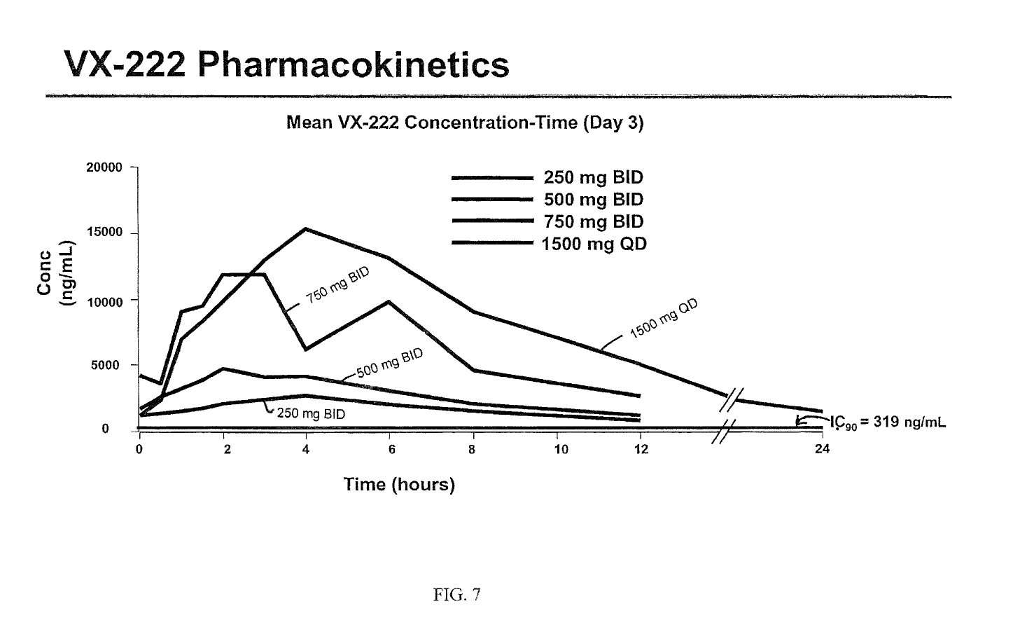

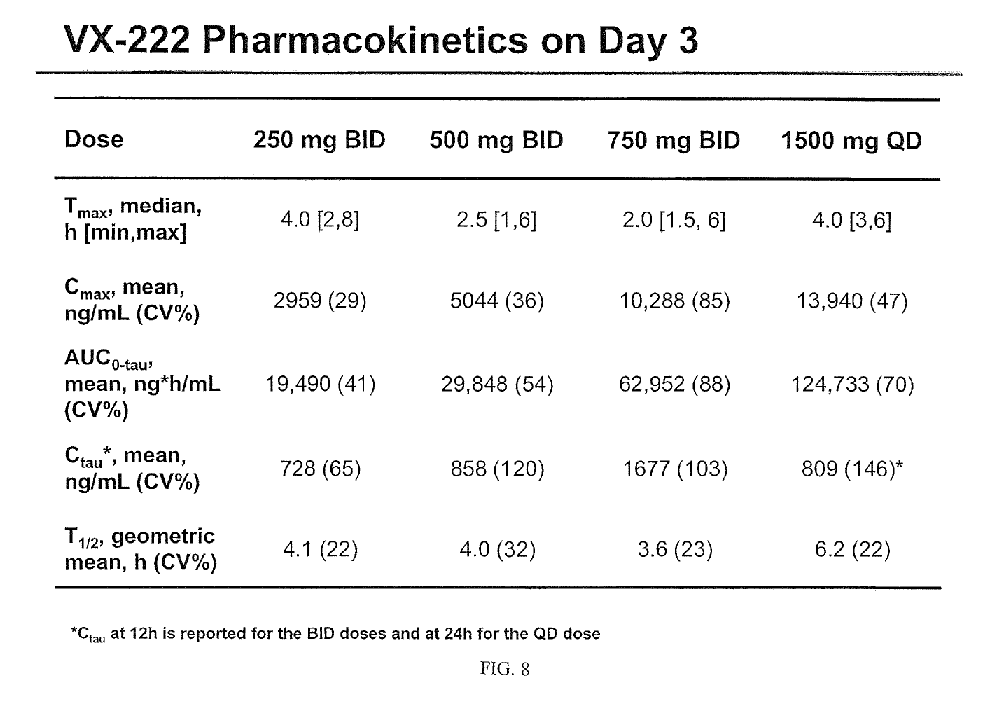

Multicenter, randomized, double-blinded, placebo-controlled, dose-ranging study
HCV genotype 1-infected patients
   With no evidence of cirrhosis
   ALT values <5×ULM
   Plasma HCV RNA of >5 log₁₀ IU/mL at screening
4-arm comparative study (6:2 randomization VX-222: placebo)
Enrollment in this dosing—ranging study was sequential
   250 mg BID for 3 days
   500 mg BID for 3 days
   750 mg BID for 3 days
   1500 mg QD for 3 days.
      Baseline characteristics are summarized in FIG. 3. HCV RNA changes are shown in FIGS. 4-6. FIG. 7 shows VX-222 pharmacokinetics. As shown in FIG. 7, Tmax was reached at 2-6 hours post does, and VX-222 exposures increased with doses in an approximately proportional manner. FIG. 8 shows a summary of VX-222 pharmacokinetics on Day 3.

### Viral Kinetic Results

Treatment with VX-222 resulted in mean reductions in plasma HCV RNA of greater than 3 log₁₀ across the four VX-222 dose groups. Additionally, an increasing dose response was observed across the four dose groups, with the results in the 500 mg, 750 mg and 1,500 mg dose groups being very similar. The mean HCV RNA decline achieved after three days of dosing with 250 mg BID, 500 mg BID, and 750 mg BID of VX-222 was 3.1 log₁₀ (range: 2.0 to 4.2), 3.4 log₁₀ (range: 3.2 to 3.6), and 3.2 log₁₀ (range: 2.3 to 3.8), respectively. The mean HCV RNA decline achieved after three days of dosing with 1,500 mg QD of VX-222 was 3.4 log₁₀ (range: 3.1 to 3.9). In the patients receiving placebo, no notable decline in HCV RNA was observed. Similar viral declines were observed for patients infected with genotype 1a and 1b.

These results of Part A of this trial are consistent with the findings from a previously conducted three-day, five-patient viral kinetic study of VX-222 dosed as 750 mg BID.

### Safety and Tolerability Results

Safety and tolerability information collected for Part A of this trial remains blinded and thus the safety information provided today includes pooled data for patients after administration of placebo or VX-222. Placebo or VX-222 were well-tolerated across all four dose groups, no severe or serious adverse events were reported and no treatment discontinuations occurred. All adverse events reported after administration of placebo or VX-222 were mild or moderate in severity. The most frequently reported adverse events occurring in at least two patients per dose group were diarrhea, headache, nausea, asthenia and fever.

### Example 8

### Pharmacokinetic Data from the Study VX-222-102 in Part C: Treatment with VX-222

In Part C of Study 101, VX-222 was administered as multiple doses of 750 mg b.i.d. to treatment-naïve subjects with chronic hepatitis C for 3 days. In addition, in Part A of Study 102, VX-222 was administered as multiple doses ranging from 250 mg to 750 mg BID and 1500 mg once a day to treatment-naïve subjects with chronic hepatitis C for 3 days.

The PK parameters of VX-222 assessed in Study 101 (final data) and Study 102 (preliminary data) are outlined below:
The accumulation index for AUC was 1.90 and for Cₘₐₓ was 1.75 fold.
Repeated measure analyses suggested that steady state was achieved within 3 days of treatment.
VX-222 exposures increased with increasing dose as indicated by the increase in mean Cₘₐₓ, AUC_{τ}, and C_{τ}. The VX-222 exposures increased in an approximately dose proportional manner in the dose ranging from 250 to 750 mg b.i.d.
VX-222 absorption was slow, with median tₘₐₓ at steady state ranging from 2 to 6 hours.
The accumulation index was around 2-fold for b.i.d. regimens. For the qd regimen, 1500 mg VX-222, exposures on Day 3 were similar to those observed on Day 1.
The mean t_{1/2} remained unchanged across all doses.
The VX-222 t₁₇₂ was around 5 hours.
VX-222 concentrations, at the end of the dosing intervals, were above the in vitro IC₉₀ (319 ng/mL) for all subjects.
In general, VCH-222 exposures in subjects were around 2-fold higher in treatment-naïve HCV subjects compared to healthy subjects.

### VX-222 Clinical Studies Efficacy in Part C

A primary objective of Part C of Study 101 was to assess the pharmacodynamics of VX-222 in treatment-naïve subjects with genotype 1 chronic hepatitis C infection. The final pharmacodynamic parameters of VX-222 administered at 750 mg b.i.d. for 3 consecutive days are outlined below:
The mean untransformed baseline (Day 1) HCV plasma RNA level was 4962600 IU/mL (log₁₀=6.4927).
The mean maximum reduction from baseline calculated using the log₁₀ predose HCV plasma RNA on Days 2 to 4 was −3.6784. The correlation between the predose RNA levels and the amplitude of the reduction in log 10 HCV RNA was relatively weak.
   These results are consistent with healthy subjects, the VX-222 administered with VX-950 was 2-fold higher than VX-222 alone.

### Example 9

### Drug Combination Assay: VX-950 and VX-222

### Materials and Methods

### Cells

Replicon cell lines Huh-7, ET cells derived from the Huh-7 hepatocarcinoma cell line are obtained from Dr. Ralf Bartenschlager (Bartenschlager, R. Innovation: Hepatitis C virus replicons: potential role for drug development. Nat. Rev. Drug Discov. 2002, 1, 911-916. Krieger, N.; Lohmann, V.; Bartenschlager, R. Enhancement of hepatitis C virus RNA replication by cell culture-adaptive mutations. J. Virol. 2001, 75, 4614-4624. Lohmann, V.; Korner, F.; Koch, J.-O.; Herian, U.; Theilmann, L.; Bartenschlager, R. Replication of subgenomic hepatitis C virus RNAs in a hepatoma cell line. Science. 1999, 285, 110-113.) (Reblikon GmbH, Gau-Odernheim, Germany). The Huh-7, ET cell line contains the highly cell culture-adapted replicon I₃₈₉luc-ubi-neo/NS3-3′/5.1 construct (characterized also by the presence of three adaptive mutations within the HCV NS3 and the NS5 genes) that carries, in addition to the neomycin gene, an integrated copy to the firefly luciferase gene (Vrolijk, J. M.; Kaul, A.; Hansen, B. E.; Lohmann, V., Haagmans, B. L.; Schalm, S. W.; Bartenschlager, R. A replicon-based bioassay for the measurement of interferons in patients with chronic hepatitis C. Journal of Virol. Methods. 2003, 110, 201-209). This cell line allows measurement of HCV RNA replication and translation by measuring luciferase activity. It has been previously shown that the luciferase activity tightly follows the replicon RNA level in these cells (J. Virol. 2001, 75, 4614-4624, Journal of Virol. Methods. 2003, 110, 201-209). The culture media used for cell culture consists of DMEM (Wisent Inc., St-Bruno, QC, Canada) supplemented with 10% foetal bovine serum with 1% penicillin/streptomycin, 1% glutamine, 1% sodium pyruvate, 1% non-essential amino acids and 180 μg/mL of Geneticin (G418) (Invitrogen, Burlington, ON, Canada) final concentrations. Cells are incubated at 37° C., in an atmosphere of 5% CO₂ and passaged twice a week to maintain sub-confluence.

Replicon cell lines Huh-7, 9-13 cells derived from the Huh-7 hepatocarcinoma cell line are obtained from Dr. Ralf Bartenschlager (Reblikon GmbH, Gau-Odemheim, Germany). The Huh-7, 9-13 cells line contains the HCV sub-genomic replicon pFK I₃₇₇/NS3-3′/wt (Koutsoudakis, G.; Kaul, A.; Steinmann, E.; Kallis, S.; Lohmann, V.; Pietschmann, T.; Bartenschlager, R. Characterization of the Early Steps of Hepatitis C Virus Infection by Using Luciferase Reporter Viruses. J. Virol. 2006, 80, 5308-5320) and is used in the real time PCR assay. The quantitative real time PCR assay (Taqman) is essentially a standard PCR in conjunction with the use of a fluorogenic oligonucleotide probe to which a reporter dye and a quencher dye are attached. During PCR, the probe anneals to the target of interest between the forward and reverse primer sites. During each extension, the probe is cleaved by the 5′ nuclease activity of the Taq DNA polymerase. This separates the reporter dye from the quencher dye, generating an increase in the reporter dye's fluorescence intensity. The intensity of fluorescence is proportional to the amount of target DNA present.

### Drug Combination Assay

Luciferase Assay (4-Day Experiment Using MacSynergy Analysts)

Huh-7, ET replicon cells are seeded in white opaque 96-well cell culture microtiter plates at sub-confluent density (3×10³ cells per well) in a volume of 100 μL media. The cell culture media used for the assay is the same as described above except that it contains neither G418 nor phenol red. A matrix of stock solutions containing a combination of thiophene and either, different anti-HCV drugs, in various concentrations prepared in deep-96-well plates. After an incubation period of 3-4 hours at 37° C., compounds (100 μL) from the matrix stock solution plates are added to the cells, whereby one compound is titrated horizontally and one compound vertically, for a final volume of 200 μL. At least four plates of cells are used for each drug-drug interaction experiment and each combination is done at least twice. Cells are then further incubated for 4 days at 37° C. in a 5% CO₂ atmosphere. Thereafter, the culture media is removed and cells are lysed by the addition of 95 μL of the Luciferase buffer (luciferin substrate in buffered detergent). Cell lysates are incubated at room temperature and protected from direct light for at least 10 minutes. Plates are read for Luciferase counts using a luminometer (Wallac MicroBeta Trilux, Perkin Elmer™, MA, USA).

In order to determine if the combinations of compounds are additive, synergistic, or antagonistic, drug combination effects of the four-days treatment between thiophene and each of the other drugs are calculated by MacSynergy II™ program (Prichard, M. N.; Prichard, L. E.; Shipman, C. Strategic design and three-dimensional analysis of antiviral drug combinations. Antimicrob. Agents Chemother. 1993, 37:540-545. Prichard, M. N. and Shipman, C. A three-dimensional model to analyze drug-drug interactions. Antiviral Res. 1990, 14:181-205). This method examines drug combinations by using the Bliss independence null model that is based on statistical probability and assumes that two drugs act independently to inhibit replication. Using this method, the theoretical additive interactions are calculated from the dose-response curves of the individual drugs acting alone. The predicted additive effects are then subtracted from the experimentally determined effects to reveal a difference in dose-response surface. The resulting surface appears as a horizontal plane at 0% difference if the interactions are additive. Any peaks above the plane are indicative of a greater-than-expected effect (synergy). Conversely, peaks appearing below the plane are indicative of a less-than-expected effect (antagonism). The confidence intervals around the experimental dose-response surface are used to evaluate the data statistically, and the volume of the peaks is calculated to quantify the synergy or antagonism produced.

The 50% inhibitory concentrations (IC₅₀s) for inhibitory effect of all drugs, when tested alone, are also determined from dose response curves using seven to nine concentrations per compound. Curves are fitted to data points using nonlinear regression analysis, and IC₅₀s are interpolated from the resulting curve using GraphPad Prism software, version 2.0 (GraphPad Software Inc., San Diego, Calif., USA):
≦25 uM²%: Insignificant amount of synergy
25 uM²-50 uM²%: Minor synergy
50 uM²%-100 uM²%: Moderated synergy
≧100 uM²%: Strong synergy.

### Real Time PCR Assay (HCV Replicon Viral RNA Clearance and Rebound Experiments).

Replicon cell lines Huh-7, 9-13 cells are seeded at a density of 3×10⁴ cells per well in a volume of 1 mL in a 12-well culture dish. The cell culture media used for the assay is DMEM supplemented with 10% fetal bovine serum, 1% penicillin/streptomycin, 1% glutamine, 1% sodium pyruvate, and 1% non-essential amino acids. After an incubation period of 3 to 4 hours, compounds are added at various concentrations for a final volume of 2 mL. Cells are then further incubated at 37° C. in a 5% CO₂ atmosphere for 14 days. The cells are split every 3 to 4 days, the media and inhibitors are replenished, and a sample of cells is harvested for RNA quantification by real time PCR. After 14 days of incubation, the cells are split and plated into fresh media in the absence of antiviral compounds. At day 18, the cells are split and plated into fresh selective media containing 0.25 mg/mL of G418 antibiotic. The culture is followed until day 42 in the presence of G418, and cells are split every 3 to 4 days where a cell sample is taken for RNA quantification by real time PCR. Total RNA (cellular and viral origin) is extracted using the Qiagen RNeasy reagent (Qiagen Inc., Mississauga, ON, Canada, kit: 74106) according to manufacturer protocol and the cDNA synthesis using the MMLV RT enzyme is performed. This step is followed by PCR reactions using appropriate oligonucleotides, Taqman probes, and DNA Taq polymerase on a ABI PRISM® 7700 Sequence Detection System (Applied Biosystems, Foster City, Calif., USA) for real-time detection of the PCR. The 18S RNA level is used to normalize for the amount of total RNA in each well.

The 50% and 90% inhibitory concentrations (IC₅₀s and IC₉₀s) for inhibitory effect of all drugs, when tested alone, are also determined from dose response curves using six concentrations in duplicate per compound. Curves are fitted to data points using nonlinear regression analysis, and IC₅₀ and IC₉₀ are interpolated from the resulting curve using GraphPad Prism software, version 2.0 (GraphPad Software Inc., San Diego, Calif., USA).

Combination Studies of Thiophene Compounds with Viral Protease Inhibitors.

Combination studies of selected thiophene compounds used the materials and methods section described above. The selected HCV NS3 protease inhibitor for the study is VX-950. The result of the volume of synergy between these compounds using the MacSynergy™ software is described in Table 4.

TABLE 4
Combination studies of thiophene compounds with HCV NS3
protease inhibitors using MacSynergy ™ software.

| | | Results |
|---|---|---|
| | | VX-950 |
| Cpd # | Thiophene structure | (Vertex) |
| 1 | 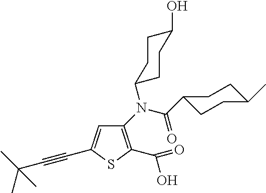 [SMILES: CC1CCC(C(=O)N(c2cc(C#CC(C)(C)C)sc2C(=O)O)C2CCC(O)CC2)CC1] | Moderated synergy (90 uM² %) (86 uM² %) |

### Example 10

### Syntheses of Certain Prodrugs of Compound 1

As used herein the term RT (min) refers to the LCMS retention time, in minutes, associated with the compound. NMR and Mass Spectroscopy data of certain specific compounds are summarized in Table 5.

### Preparation of Compound A

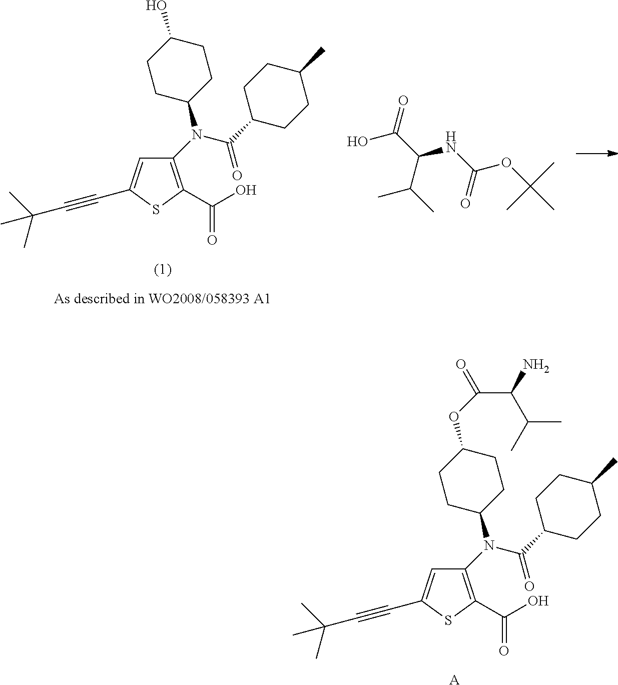

5-(3,3-Dimethylbut-1-ynyl)-3-[(trans-4-hydroxycyclohexyl)-(4-trans methylcyclohexanecarbonyl)amino]thiophene-2-carboxylic acid (compound (1), 300 mg, 0.67 mmol) was dissolved in dichloromethane (DCM, 15 mL). To this was added (2S)-2-(tert-butoxycarbonylamino)-3-methyl-butanoic acid Boc-L-valine (176 mg, 0.81 mmol), N,N-dimethylpyridin-4-amine (DMAP, 8.22 mg, 0.067 mmol), triethylamine (Et₃N, 136 mg, 187 μL, 1.35 mmol), and 3-(ethyliminomethyleneamino)-N,N-dimethyl-propan-1-amine hydrochloride (EDC, 129 mg, 0.67 mmol). The reaction was stirred overnight. The reaction mixture was then concentrated, diluted with ethyl acetate (EtOAc), washed with water, and the combined organic layers washed with brine and dried with sodium sulfate. Filtration and concentration gave a yellow oil, which was purified by column chromatography. The resulting product was then treated with 4N HCl in dioxane (15 mL) to give the desired compound A as the HCl salt (100 mg, 26%): MS: m/z (obs.): 545.4 [M+H]⁺; Retention time: 3.45 min; 1H NMR (300 MHz, MeOH) δ 7.04 (s, 1H), 4.75-4.58 (m, 1H), 4.39 (dt, J=14.5, 9.4 Hz, 1H), 3.85 (d, J=4.4 Hz, 1H), 3.80-3.68 (m, 1H), 3.61-3.51 (m, 1H), 2.24 (dt, J=14.0, 6.9 Hz, 1H), 2.01 (dd, J=15.2, 7.3 Hz, 6H), 1.60 (dd, J=28.5, 14.8 Hz, 9H), 1.34 (s, 9H), 1.18-0.99 (m, 3H), 0.81 (d, J=6.5 Hz, 3H), 0.66 (dd, J=25.3, 12.9 Hz, 1H).

### Preparation of Compound B

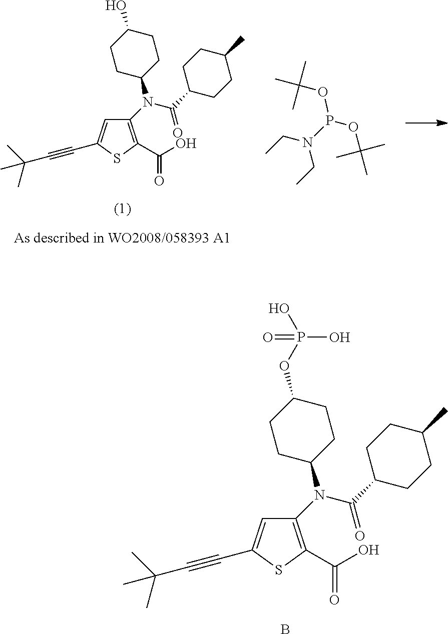

5-(3,3-Dimethylbut-1-ynyl)-3-[(trans 4-hydroxycyclohexyl)-(trans 4-methylcyclohexanecarbonyl)amino]thiophene-2-carboxylic acid (compound (1), 100 mg, 0.12 mmol) was dissolved in dichloromethane (DCM, 10.0 mL) and cooled to 0° C. Tetrazole (4.0 mg, 0.058 mmol) was added followed by N-(di-tert-butoxyphosphanyl)-N-ethyl-ethanamine (288 mg, 322 μL, 1.16 mmol). The reaction was stirred overnight at room temperature, then cooled to −78° C. 3-Chlorobenzenecarboperoxoic acid (MCPBA) (99.7 mg, 0.58 mmol) was added and the reaction stirred for 2 hours then quenched with aq. Na₂SO₃. The mixture was extracted with ethyl acetate and the extracts washed with water. The organic layer was concentrated to give a colorless oil, which was purified by ISCO silica gel chromatography and taken directly to the next step. To the product was added CH₂Cl₂ (5 mL) and 2,2,2-trifluoroacetic acid (TFA) (5 mL). The reaction was stirred for 2 hours, then concentrated and the product B purified by HPLC: MS: m/z (obs.): 526.39 [M+H]⁺; Retention time: 6.51 min; ¹H NMR (300 MHz, d6-DMSO) δ 7.18 (s, 1H), 4.29 (t, J=11.8 Hz, 1H), 3.83 (s, 1H), 2.53 (d, J=8.2 Hz, 3H), 1.84 (s, 2H), 1.75-1.33 (m, 7H), 1.30 (s, 9H), 1.27-1.09 (m, 3H), 0.90 (d, J=12.9 Hz, 2H), 0.76 (d, J=6.5 Hz, 2H), 0.70-0.47 (m, 2H); ³¹P NMR (121.5 MHz, d6-DMSO) δ −2.01 (s).

### Preparation of Compound C

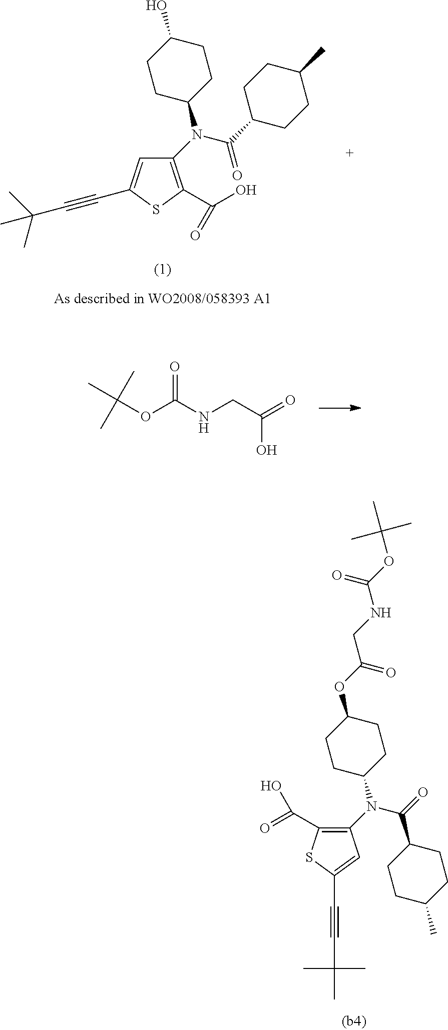

To a solution of 5-(3,3-dimethylbut-1-ynyl)-3-[(4-trans-hydroxycyclohexyl)-(4-trans-methylcyclohexanecarbonyl)amino]thiophene-2-carboxylic acid (compound (1), 75 mg, 0.17 mmol) and N-Boc-glycine (44.2 mg, 0.25 mmol) in CH₂Cl₂ (15 mL) was added 3-(ethyliminomethyleneamino)-N,N-dimethyl-propan-1-amine hydrochloride (EDC) (32.2 mg, 0.17 mmol), N,N-dimethylpyridin-4-amine (DMAP) (10.3 mg, 0.084 mmol) and Et₃N (34 mg, 0.33 mmol). The reaction mixture was stirred at ambient temperature overnight then the reaction mixture was evaporated and purified by ISCO silica gel chromatography to give compound (b4), [O—(N-t-Butoxycarbonyl)-glycyl]-5-(3,3-dimethylbut-1-ynyl)-3-[(4-trans-hydroxycyclohexyl)-(4-trans-methylcyclohexanecarbonyl)amino]thiophene-2-carboxylic acid: MS: m/z (obs.): 603.17 [M+H]⁺; Retention time: 2.31 min.

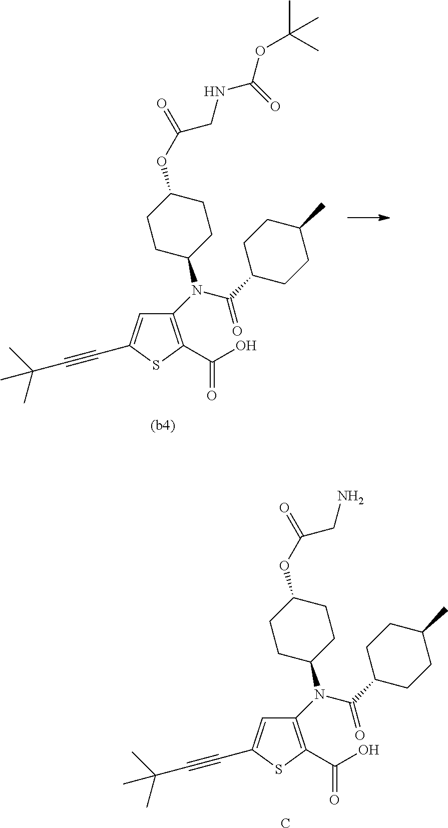

[O—(N-t-Butoxycarbonyl)-glycyl]-5-(3,3-dimethylbut-1-ynyl)-3-[(4-trans-hydroxycyclohexyl)-(4-trans-methylcyclohexanecarbonyl)amino]thiophene-2-carboxylic acid (Compound (b4), 40 mg, 0.066 mmol) was treated with 4N HCl in dioxane (1 mL) and stirred at RT overnight. Then the reaction mixture was concentrated and purified by HPLC to give compound C (11 mg): MS: m/z (obs.): 503.35 [M+H]⁺; Retention time: 2.24 min.

### Preparation of Compound D

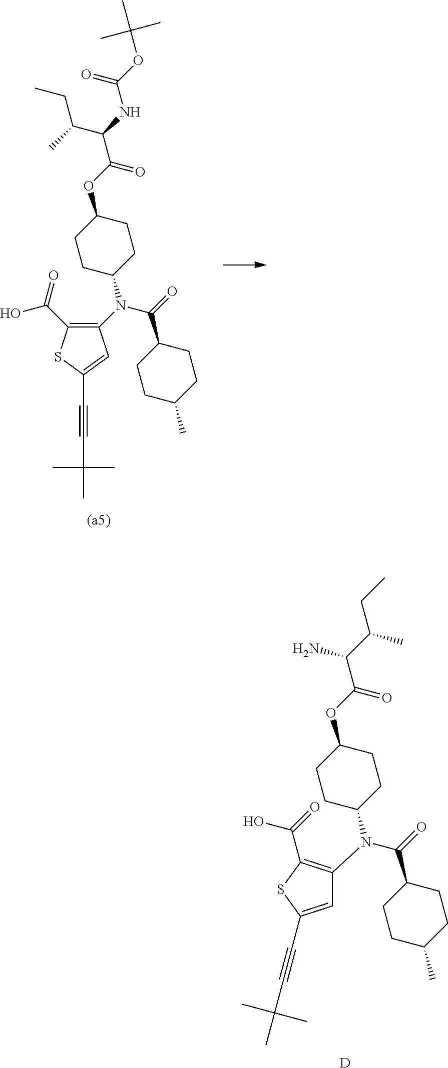

Compound (a5), [O—(N-t-Butoxycarbonyl)-D-isoleucyl]-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-D-isoleucine as described for Compounds 1 & 4 above) was treated with 4N HCl in dioxane (10 mL) and stirred at RT overnight. Then the reaction mixture was concentrated and purified by HPLC to give compound D: MS: m/z (obs.): 559.4 [M+H]⁺; Retention time: 2.39 min.

### Preparation of Compound E

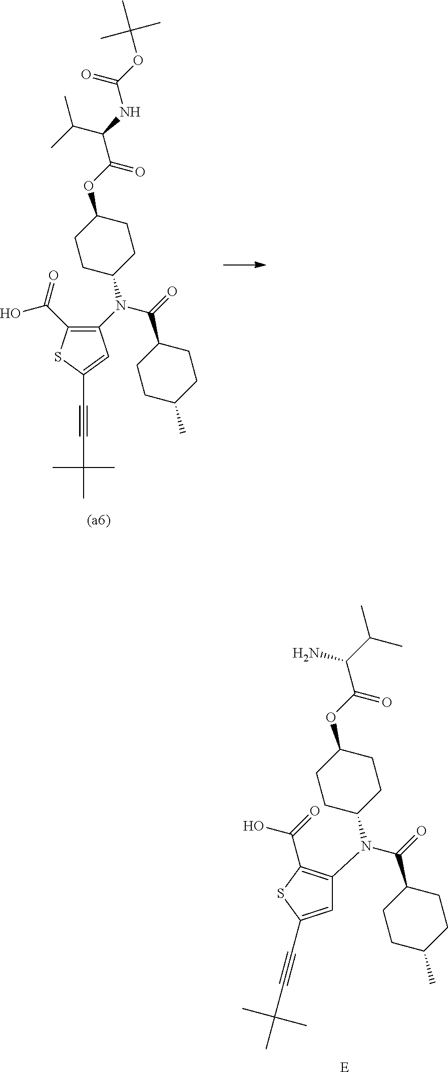

Compound (a6), [O—(N-t-Butoxycarbonyl)-D-valinyl]-5-(3,3-dimethylbut-1-ynyl)-3-[(4-trans-hydroxycyclohexyl)-(4-trans-methylcyclohexanecarbonyl)amino]thiophene-2-carboxylic acid (30 mg) (prepared from Boc-D-valine as described for Compounds 1 & 4 above) was treated with 4N HCl in dioxane (10 mL) and stirred at RT overnight. Then the reaction mixture was concentrated and purified by HPLC to give compound E: MS: m/z (obs.): 545.39 [M+H]⁺; Retention time: 2.35 min.

### Preparation of Compound F

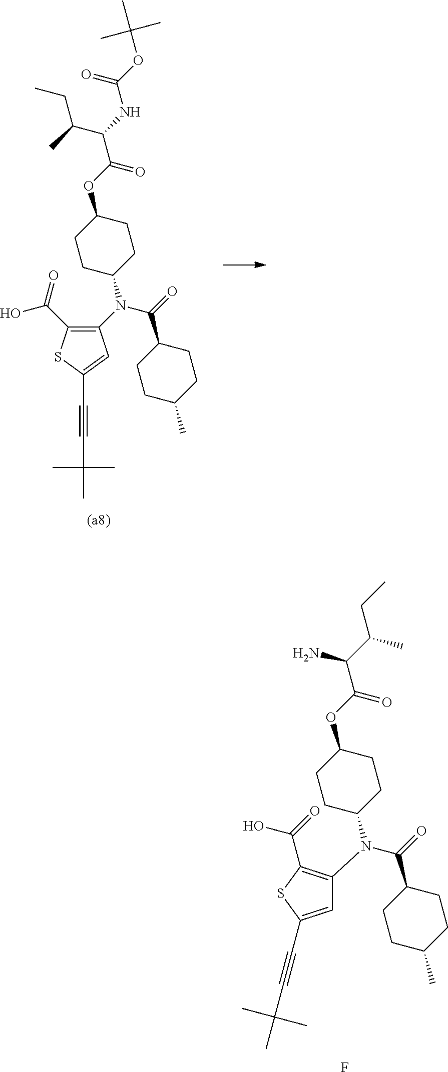

Compound (a8), (O—(N-t-Butoxycarbonyl)-L-isoleucyl)-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-L-isoleucine as described for Compounds A & C above) (35 mg) was treated with 4N HCl in dioxane (10 mL) and stirred at RT for overnight. Then the reaction mixture was concentrated and purified by HPLC to give compound 8: MS: m/z (obs.): 559.47 [M+H]⁺; Retention time: 3.2 min.

### Preparation of Compound G

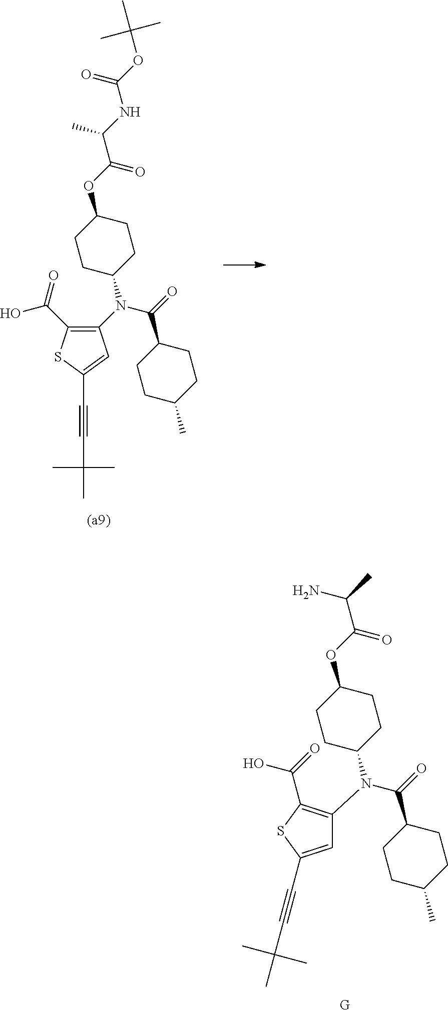

Compound (a9), (O—(N-t-Butoxycarbonyl)-L-alanyl)-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-L-alanine as described for Compounds A & C above) (25 mg) was taken in 4N HCl in dioxane and stirred at RT overnight. Then the reaction mixture was concentrated and purified by HPLC to give compound G: MS: m/z (obs.): 517.43 [M+H]⁺; Retention time: 2.99 min.

### Preparation of Compound H

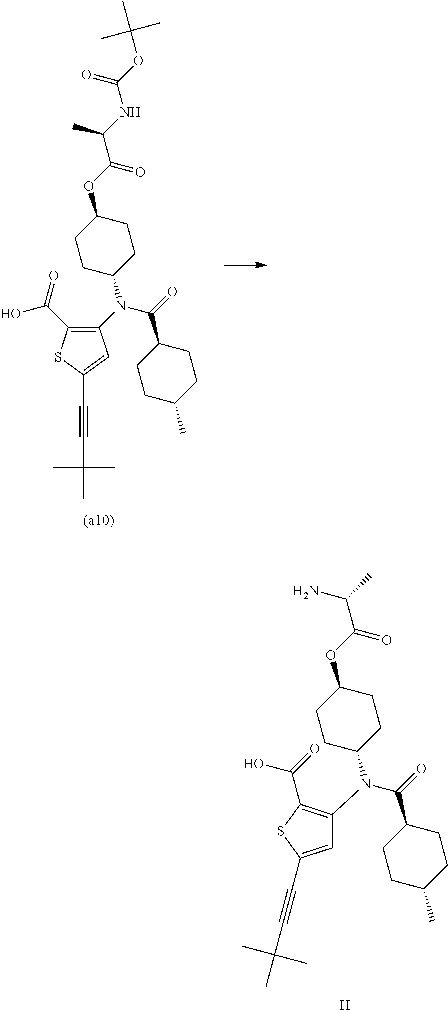

Compound (a10), (O—(N-t-Butoxycarbonyl)-D-alanyl)-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-D-alanine as described for Compounds 1 & 4 above) (35 mg, 0.058 mmol) was treated with 4N HCl in dioxane (10 mL) and stirred at RT overnight. Then the reaction mixture was concentrated and purified by HPLC to give compound H: MS: m/z (obs.): 517.43 [M+H]⁺; Retention time: 3.0 min.

TABLE 5
LCMS and NMR data of Compounds A-H (Prodrugs of Compound 1)

| | LCMS | LCMS | |
|---|---|---|---|
| Compounds | [M + H]⁺ | RT | NMR |
| A | 545.45 | 3.45 | 1H NMR (300 MHz, MeOH) δ 7.04 (s, 1H), |
| | | | 4.75 − 4.58 (m, 1H), 4.39 (dt, J = 14.5, 9.4 Hz, 1H), |
| | | | 3.85 (d, J = 4.4 Hz, 1H), 3.80 − 3.68 (m, 1H), |
| | | | 3.61 − 3.51 (m, 1H), 2.24 (dt, J = 14.0, 6.9 Hz, 1H), |
| | | | 2.01 (dd, J = 15.2, 7.3 Hz, 6H), 1.60 (dd, J = 28.5, |
| | | | 14.8 Hz, 9H), 1.34 (s, 9H), 1.18 − 0.99 (m, 3H), 0.81 |
| | | | (d, J = 6.5 Hz, 3H), 0.66 (dd, J = 25.3, 12.9 Hz, 1H). |
| B | 526.39 | 6.51 | ¹H NMR (300 MHz, d6-DMSO) δ 7.18 (s, 1H), 4.29 |
| | | | (t, J = 11.8 Hz, 1H), 3.83 (s, 1H), 2.53 (d, J = 8.2 Hz, |
| | | | 3H), 1.84 (s, 2H), 1.75 − 1.33 (m, 7H), 1.30 (s, 9H), |
| | | | 1.27 − 1.09 (m, 3H), 0.90 (d, J = 12.9 Hz, 2H), |
| | | | 0.76 (d, J = 6.5 Hz, 2H), 0.70 − 0.47 (m, 2H); |
| | | | ³¹P NMR (121.5 MHz, d6-DMSO δ −2.01 (s). |
| C | 503.35 | 2.24 | |
| D | 559.4 | 2.39 | |
| E | 545.39 | 2.35 | |
| F | 559.47 | 3.2 | |
| G | 517.43 | 2.99 | |
| H | 517.43 | 3 | |

### Example 10

### PK Parameters of Prodrugs of Compound 1

The prodrugs whose PK parameters are to be determined can be formulated as a solution in 0.5% MC/0.5% Tween 80/99% water and administered orally by gavage to rats at a dose of 3 mg/kg. Rats are weighed the day before the study. Rat plasma is sampled predose and at 15, 30 min, 1, 2, 3, 4, 6, 8, 12 and 24 hrs post dose using Instech automatic blood sampling equipment. Blood is collected in tubes containing K2-EDTA and 110 uL plasma are extracted for analysis. Rats are fed ad lib and standard IACUC and SOP protocols are followed. Plasma samples and dose samples are analyzed using LC/MS/MS for both the prodrug compound and the active metabolite. PK parameters for both analytes for each subject are calculated using the measured dose of prodrug.

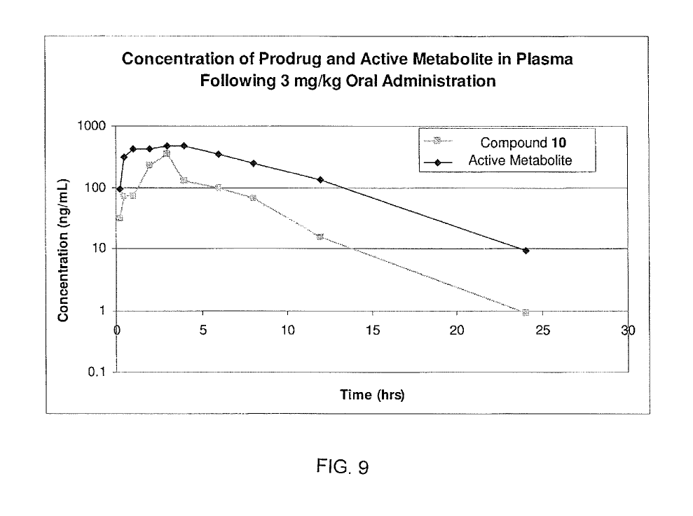

The PK parameters of Compound H (designated as “Compound 10” in FIG. 9, a prodrug of Compound 1) was measured as described in the preceding paragraph, and are depicted in FIG. 9. As shown in FIG. 9, the —O-alaninyl group of compound H in vivo converted into the —OH active metabolite.

What is claimed is:

1. A method of improving the pharmacokinetics of VX-222 in a patient infected with HCV, comprising co-administering VX-222 and VX-950 to the patient.

2. The method of claim 1, wherein exposure of VX-222 in the plasma, blood, or liver of the patient is improved.

3. (canceled)

4. The method of claim 1, wherein the exposure of VX-222 in the plasma of the patient is increased by a two-six fold compared to the plasma exposure of VX-222 when administered without VX-950.

5. (canceled)

6. The method of claim 1, wherein a C(trough) level of VX-222 is increased.

7. The method of claim 1, wherein a C(max) value of VX-222 is increased.

8. The method of claim 1, wherein an AUC value of VX-222 is increased.

9. The method of claim 1, wherein VX-222 is administered in an amount of about 20 mg to about 2,000 mg at each administration.

10. (canceled)

11. (canceled)

12. (canceled)

13. The method of claim 9, wherein VX-222 is in an amount of about 400 mg at each administration.

14. (canceled)

15. (canceled)

16. (canceled)

17. (canceled)

18. The method of claim 1, wherein VX-222 is administered twice a day.

19. (canceled)

20. (canceled)

21. (canceled)

22. (canceled)

23. (canceled)

24. The method of claim 1, wherein VX-950 is administered at about 750 mg three times per day.

25. (canceled)

26. The method of claim 1, wherein VX-950 is administered at about 1,125 mg two times per day.

27. The method of claim 1, further comprising administering one or more additional HCV drugs other than VX-950 and VX-222 to the patient.

28. The method of claim 27, wherein an interferon is co-administered.

29. (canceled)

30. (canceled)

31. The method of claim 27, wherein the interferon is pegylated-interferon alpha-2a or pegylated-interferon alpha-2b.

32. The method of claim 27, wherein ribavirin is co-administered.

33. The method of claim 1, wherein VX-950 and VX-222 are coadministered for a period of time in a range of about 8 weeks to about 24 weeks.

34. The method of claim 33, wherein VX-950 and VX-222 are co-administered for about 12 weeks.

35. (canceled)

36. (canceled)

37. (canceled)

38. (canceled)

39. (canceled)

40. A method of treating a patient infected with HCV, comprising administering VX-222 and VX-950 to the patient, wherein VX-222 is in an amount of about 20 mg to about 400 mg at each administration, and wherein VX-950 is in an amount of about 100 mg to about 1500 mg at each administration.

41. (canceled)

42. (canceled)

43. (canceled)

44. The method of claim 40, wherein VX-950 is administered at about 750 mg three times per day.

45. The method of claim 40, wherein VX-950 is administered at about 1,125 mg two times per day.

46. (canceled)

47. The method of claim 40, wherein VX-222 is in an amount of about 400 mg at each administration.

48. (canceled)

49. The method of claim 40, wherein VX-222 is administered twice a day.

50. The method of claim 40, further comprising administering one or more additional HCV drugs other than VX-950 and VX-222 to the patient.

51. The method of claim 50, wherein an interferon is co-administered.

52. (canceled)

53. (canceled)

54. The method of claim 51, wherein the interferon is pegylated-interferon alpha-2a or pegylated-interferon alpha-2b.

55. The method of claim 50, wherein ribavirin is co-administered.

56. The method of claim 40, wherein VX-950 and VX-222 are administered for a period of time about 8 weeks to about 24 weeks.

57. The method of claim 56, wherein VX-950 and VX-222 are administered for about 12 weeks.

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. A pharmaceutically acceptable composition, comprising:
a) VX-222 in an amount of about 20 mg to about 400 mg; and
b) VX-950 is in an amount of about 100 mg to about 1,500 mg.

89. (canceled)

90. The composition of claim 88, wherein VX-222 in an amount of about 100 mg to about 400 mg; and VX-950 is in an amount of about 300 mg to about 750 mg.

91. The composition of claim 88, wherein VX-950 is in an amount of about 375 mg.

92. (canceled)

93. The composition of claim 88, wherein VX-222 in an amount of about 400 mg.
